(19) United States
(12) Patent Application Publication
(10) Pub. No.: US 2025/0032149 A1
(43) Pub. Date: Jan. 30, 2025

(54) ATHERECTOMY CATHETER WITH SHAPEABLE DISTAL TIP

(71) Applicant: Avinger, Inc., Redwood City, CA (US)

(72) Inventor: Himanshu N. PATEL, San Jose, CA (US)

(21) Appl. No.: 18/716,831

(22) PCT Filed: Dec. 6, 2022
(86) PCT No.: PCT/US2022/080985

§ 371 Date: Jun. 5, 2024

Related U.S. Application Data
(60) Provisional application No. 63/265,023, filed on Dec. 6, 2021.

(51) Int. Cl. A61B 17/3207; A61B 17/00

(52) U.S. Cl. CPC A61B 17/320783; A61B 2017/00057; A61B 2017/320791

(57) ABSTRACT

An atherectomy catheter for use in a vessel includes a catheter and a rotatable cutter. The rotatable cutter can be translatable within the catheter to extend the cutter through a window of the catheter or retracted the cutter within the catheter. The catheter can have a fixed bend and/or have a shapeable portion configured to facilitate positioning and movement of the cutter. The shapeable portion may take on a pre-defined shape. The shapeable portion may be positioned against an inner wall of a blood vessel to provide leverage for the rotatable cutter. In some cases, a rotation limiter may limit the number of rotations that the catheter can rotate with respect to a guidewire to prevent twisting of the guidewire.

### CROSS REFERENCE TO RELATED APPLICATIONS

This application claims priority to U.S. Provisional Application No. 63/265,023, entitled “ATHERECTOMY CATHETER WITH SHAPEABLE DISTAL TIP,” filed on Dec. 6, 2021, which is incorporated by reference herein in its entirety.

This application may also be related to International Patent Application No. PCT/US2022/019075, filed Mar. 7, 2022, titled “OCCLUSION-CROSSING DEVICES,” and published as International Patent Publication No. WO 2022/192102, which is incorporated herein by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are devices for treatment of an occluded body lumen, such as for the removal of occlusive materials from blood vessels. In particular, described herein are atherectomy catheters that are adapted to easily maneuver against tissue and plaque buildup within vessels for debulking.

### BACKGROUND

Atherosclerosis is a disease in which accumulation of atheromatous materials builds up inside a person's arteries. Atherosclerosis occurs as part of the natural aging process, but may also occur due to a person's diet, hypertension, vascular injury, heredity, and so forth. Atherosclerosis can affect any artery in the body, including arteries in the heart, brain, arms, legs, pelvis, and kidneys. Atherosclerosis deposits may vary in their properties as well. Some deposits are relatively soft, other types may be fibrous, some are calcified, or a combination of all three. Based on the location of the plaque accumulation, different diseases may develop. For example, coronary heart disease occurs when plaque builds up in the coronary arteries, which supply oxygenated blood to the heart. If plaque buildup blocks the carotid artery, arteries located on each side of the neck that supply oxygen to the brain, a stroke may be the result.

Atherosclerosis may be treated in a number of ways including medication, bypass surgery, and catheter-based approaches. Atherectomy procedures involve excising or dislodging materials that block a blood vessel. Many atherectomy catheters typically have a substantially straight central axis. However, atherectomy catheters having a straight profile may be difficult to maneuver close enough to the inner surface of the arterial walls to remove all plaque buildup. Moreover, plaque removal can be complicated with such straight profile catheters when plaque formations accumulate in the curves and more tortuous portions of an artery.

The atherectomy catheters described herein address some of these challenges.

### SUMMARY OF THE DISCLOSURE

Described herein are atherectomy catheters for use in vessels. The catheters can include a rotatable cutter within a catheter. The shape of the catheter can be configured to aid optimal positioning of the cutter, for example during a cutting procedure. In some cases, the cutter may be extended through a window of the catheter upon translation of the cutter within the catheter. In some cases, the cutter is retractable into the catheter.

One aspect of the disclosure is an atherectomy device comprising: a catheter including a distal nosecone coupled to an elongate body, the catheter including a cutter window between the nosecone and the elongate body, wherein the elongate body includes a shapeable section including a first portion, a second portion, and a third portion each configured to bend upon activation; and a driveshaft configured to rotate and translate within the catheter, the driveshaft including a distal cutter configured to extend through the cutting window, wherein a force applied to the driveshaft in a proximal direction causes the shapeable section to take on a U-shape defined by a first curve of the first portion, a second curve of the second portion, and a third curve of the third portion.

In this aspect, the nosecone may be fixedly coupled to the elongate body at a fixed bend of the catheter.

In this aspect, the cutter window may be on a convex side of the fixed bend.

In this aspect, the cutter window may be distally located along the catheter with respect to the fixed bend.

In this aspect, an extent of curvature of the shapeable section may be adjustable based on an amount of force applied to the rotatable driveshaft in the proximal direction.

In this aspect, the cutter may be configured to tilt in a first direction and extend though the cutter window upon proximal movement of the driveshaft with respect to the catheter.

In this aspect, the cutter may be configured to tilt in a second direction opposite the first direction and retract within the cutter window upon distal movement of the driveshaft with respect to the catheter.

In this aspect, the cutter may be configured to extend distally within the nosecone upon further distal movement of the driveshaft with respect to the catheter.

In this aspect, the shapeable section may be configured to revert back to a straight configuration upon distal movement of the driveshaft.

In this aspect, the shapeable section may include a frame configured to limit an extent to which the shapeable section bends.

In this aspect, the shapeable section may include a first axial portion, a second axial portion, and a third axial portion, wherein the first and third axial portions are configured to bend in a first direction, wherein the second axial portion is configured to bend in a second direction opposite the first direction.

In this aspect, each of the first, second and third axial portions may include a longitudinal backbone, wherein activation of the shapeable section causes each of the first, second and third axial portions to bend away from its corresponding backbone.

In this aspect, the shapeable section may include a tubular frame having a plurality of slits, wherein activation of the shapeable section causes compression of the tubular frame such that spaces between the plurality of slits become closer together.

In this aspect, the slits may be slanted with respect to a transverse axis of the tubular frame.

In this aspect, the shapeable section may include a tubular frame having bend control features configured to limit an extent to which the shapeable section bends.

In this aspect, the first, second and third curves may be arranged along a plane.

In this aspect, the shapeable section may include portions that are configured to bend in at least two different lateral directions.

In this aspect, the cutter may include an imaging sensor configured to collect images outside of the catheter while the cutter rotates.

One aspect of the disclosure is a method of using an atherectomy device, the atherectomy device including a cutter coupled to a rotatable driveshaft within a catheter, the catheter having a distal nosecone coupled to an elongate body and a cutter window between the distal nosecone and the elongate body, the method comprising: applying an axial force in a proximal direction on the driveshaft within the catheter to cause a shapeable section of the elongate body to form a U-shape defined by a first curve, a second curve and a third curve.

In this aspect, the method may further comprise moving the driveshaft in a proximal direction to cause the cutter to tilt in a first direction and extend through the cutter window.

In this aspect, the method may further comprise moving the driveshaft in a distal direction to cause the cutter to tilt in a second direction opposite the first direction and to retract within the catheter.

In this aspect, the method may further comprise moving the driveshaft in the distal direction to cause the cutter to extend distally within the nosecone.

In this aspect, method may further comprise selecting an extent of curvature of the shapeable section by controlling an amount of the axial force applied to the driveshaft.

In this aspect, the nosecone may be fixedly coupled to the elongate body at a fixed bend of the catheter.

In this aspect, the cutter window may be on a convex side of the fixed bend.

In this aspect, the cutter window may be distally located along the catheter with respect to the fixed bend.

In this aspect, the method may further comprise applying an axial force in a distal direction on the driveshaft within the catheter to cause the shapeable section to take on a substantially straight configuration.

In this aspect, the method may further comprise applying an axial force in a distal direction on the driveshaft within the catheter to cause the shapeable section to cause the shapeable section to bend in an opposite direction.

In this aspect, the shapeable section may include first, second and third axial portions, wherein each of the first, second and third axial portions includes a longitudinal backbone, wherein bending of the shapeable section causes each of the first, second and third axial portions to bend away from its corresponding backbone.

In this aspect, the shapeable section may include a tubular frame having a plurality of slits, wherein bending of the shapeable section causes compression of the tubular frame such that spaces between the plurality of slits become closer together.

In this aspect, the slits may be slanted with respect to a transverse axis of the tubular frame.

In this aspect, the shapeable section may include a tubular frame having bend control features configured to limit an extent to which the shapeable section bends in a first lateral direction and a second lateral direction.

In this aspect, the first, second and third curves may be along a plane.

In this aspect, the method may further comprise: pressing at least one of the first, second and third curves on an inner surface of a blood vessel of a patient; and rotating the cutter to cut tissue, wherein pressing the at least one of the first, second and third curves on the inner surface of a blood vessel provides an opposing force for efficient cutting of the tissue.

In this aspect, the method may further comprise collecting images outside of the catheter using an imaging sensor coupled to the cutter as the cutter rotates.

One aspect of the disclosure is an atherectomy device comprising: a catheter having a lumen and a cutter window providing access to the lumen, wherein the catheter includes a shapeable section; and a driveshaft configured to rotate and translate within the lumen of the catheter, the driveshaft including a distal cutter configured to extend through the cutting window, wherein a force applied to the driveshaft in a proximal direction causes the shapeable section to change shape from an uncurved configuration to a curved configuration, wherein the shapeable section is axially aligned along a long axis when in the uncurved configuration, and wherein the shapeable section is curved in multiple planes relative to the long axis when in the curved configuration.

In this aspect, the shapeable section may have a spiral shape when in the curved configuration.

In this aspect, the shapeable section may be configured to bend such that the spiral shape twists up to 180 degrees.

In this aspect, the shapeable section may be configured to bend such that the spiral shape twists up to 270 degrees.

In this aspect, the shapeable section may be configured to bend such that the spiral shape twists up to 360 degrees.

In this aspect, the catheter may include a distal nosecone coupled to an elongate body, wherein the shapeable section is part of the elongate body.

In this aspect, the nosecone may be fixedly coupled to the elongate body at a fixed bend of the catheter.

In this aspect, the cutter window may be on a convex side of the fixed bend.

In this aspect, the cutter window may be distally located along the catheter with respect to the fixed bend.

In this aspect, an extent of curvature of the shapeable section may be adjustable based on an amount of force applied to the rotatable driveshaft in the proximal direction.

In this aspect, the cutter may be configured to tilt in a first direction and extend though the cutter window upon proximal movement of the driveshaft with respect to the catheter.

In this aspect, the cutter may be configured to tilt in a second direction opposite the first direction and retract within the cutter window upon distal movement of the driveshaft with respect to the catheter.

In this aspect, the cutter may be configured to extend distally with in a distal nosecone upon further distal movement of the driveshaft with respect to the catheter.

In this aspect, the shapeable section may include a frame configured to limit an extent to which the shapeable section bends in the curved configuration.

In this aspect, the shapeable section may include a first axial portion connected by a junction region by a second axial portion, wherein the first and second axial portions are configured to twist in same direction.

In this aspect, each of the first and second axial portions may include a longitudinal backbone.

In this aspect, the shapeable section may include a tubular frame having a plurality of slits, wherein activation of the shapeable section causes compression of the tubular frame such that spaces between the plurality of slits become closer together.

In this aspect, the shapeable section may include a tubular frame having bend control features configured to limit an extent to which the shapeable section bends.

In this aspect, the cutter may include an imaging sensor configured to collect images outside of the catheter while the cutter rotates.

One of the aspects of the disclosure is a method of using an atherectomy device, the atherectomy device including a cutter coupled to a rotatable driveshaft within a lumen of a catheter, the catheter having a cutter window, the method comprising: applying an axial force in a proximal direction on the driveshaft within the catheter to cause a shapeable section of the catheter to change shape from an uncurved configuration to a curved configuration, wherein the shapeable section is axially aligned along a long axis when in the uncurved configuration, and wherein the shapeable section is curved in multiple planes relative to the long axis when in the curved configuration.

In this aspect, the shapeable section may have a spiral shape when in the curved configuration.

In this aspect, the method may further comprise selecting an extent to which the spiral shape twists.

In this aspect, the shapeable section may be configured to bend such that the spiral shape twists up to 180 degrees.

In this aspect, the shapeable section may be configured to bend such that the spiral shape twists up to 270 degrees.

In this aspect, the shapeable section may be configured to bend such that the spiral shape twists up to 360 degrees.

In this aspect, the method may further comprise moving the driveshaft in a proximal direction to cause the cutter to tilt in a first direction and extend through the cutter window.

In this aspect, the method may further comprise moving the driveshaft in a distal direction to cause the cutter to tilt in a second direction opposite the first direction and to retract within the catheter.

In this aspect, the method may further comprise further moving the driveshaft in the distal direction to cause the cutter to extend distally within a distal nosecone.

In this aspect, the shapeable section may be part of an elongate body of the catheter, wherein the elongate body is fixedly coupled to the distal nosecone.

In this aspect, the nosecone may be fixedly coupled to the elongate body at a fixed bend of the catheter.

In this aspect, the method may further comprise applying an axial force in a distal direction on the driveshaft within the catheter to cause the shapeable section to take on a substantially straight configuration.

In this aspect, the method may further comprise an applying axial force in a distal direction on the driveshaft within the catheter to cause the shapeable section to cause the shapeable section to twist in an opposite direction.

In this aspect, the shapeable section may include first and second axial portions each including a longitudinal backbone.

In this aspect, the shapeable section may include a tubular frame having a plurality of slits, wherein bending of the shapeable section causes compression of the tubular frame such that spaces between the plurality of slits become closer together.

In this aspect, the shapeable section may include a tubular frame having bend control features configured to limit an extent to which the shapeable section bends.

In this aspect, the method may further comprise: pressing at least a portion of the shapeable section when in the curved configuration on an inner surface of a blood vessel of a patient; and rotating the cutter to cut tissue, wherein pressing the at least a portion of the shapeable section on the inner surface of a blood vessel provides an opposing force for efficient cutting of the tissue.

In this aspect, the method may further comprise collecting images outside of the catheter using an imaging sensor coupled to the cutter as the cutter rotates.

One of the aspects of the disclosure is an atherectomy device comprising: a catheter including a distal nosecone coupled to an elongate body, wherein the nosecone includes a guidewire lumen configured to accommodate a guidewire; a driveshaft configured to rotate and translate within the catheter, the driveshaft including a distal cutter; and a handle having a rotatable knob configured to rotate the catheter relative to a body of the handle, the rotatable knob including a rotation limiter configured to limit a number of rotations that the knob and catheter rotate.

In this aspect, the guidewire lumen may be offset with respect to a central axis of the nosecone.

In this aspect, the nosecone may include a reservoir for storing tissue.

In this aspect, the rotation limiter may include a nut configured to translate within an internal track of the knob, wherein rotation of the knob causes the nut to rotate within the knob and translate along the track, wherein the track includes a first stop and a second stop that are configured limit an extent of proximal and distal translation of the nut, thereby limiting the number of rotations of the knob and the catheter.

In this aspect, the knob may be operationally coupled to a disk assembly having a series of rotationally linked disks, wherein the disk assembly is configured to limit an extent of rotation of each of the disks of the disk assembly, thereby limiting the number of rotations of the knob and the catheter.

In this aspect, the rotation limiter may be configured to limit the number or rotations that the catheter rotates to no more than four rotations.

In this aspect, the guidewire lumen may include the guidewire therein, wherein the rotation limiter is configured to limit the number of rotations that the catheter rotates relative to the guidewire within the guidewire lumen.

In this aspect, the guidewire lumen may run longitudinally along the nosecone but not along the elongate body.

In this aspect, the rotation limiter may be configured to provide tactile, audible, or tactile and audible feedback to a user rotating the knob.

In this aspect, the knob may be at a distal end of the handle.

In this aspect, the catheter may include a cutter window between the nosecone and the elongate body, wherein the driveshaft includes a distal cutter configured to extend through the cutting window.

In this aspect, the nosecone may be fixedly coupled to the elongate body at a fixed bend of the catheter.

In this aspect, the cutter window may be on a convex side of the fixed bend.

In this aspect, the cutter window may be distally located along the catheter with respect to the fixed bend.

In this aspect, the nosecone may be pivotally coupled to the elongate body.

In this aspect, the elongate body may include a shapeable section configured to bend when a force is applied to the driveshaft in a proximal direction.

In this aspect, the shapeable section may include a frame having at least one backbone.

One aspect of the disclosure is a method of using an atherectomy device, the atherectomy device including a cutter coupled to a rotatable driveshaft within a catheter, the catheter having a nosecone coupled to an elongate body, the method comprising: inserting the catheter within blood vessel, wherein the nosecone includes a guidewire lumen with a guidewire therein; rotating the catheter within the blood vessel by rotating a knob of a handle at a proximal end of the catheter, wherein the knob includes a rotation limiter that limits a number of rotations that the knob and catheter rotate.

In this aspect, the guidewire lumen may be offset with respect to a central axis of the nosecone.

In this aspect, the method may further comprise cutting tissue within the blood vessel using the cutter rotated by the driveshaft.

In this aspect, the method may further comprise distally moving the cutter within the nosecone to pack tissue within a reservoir of the nosecone.

In this aspect, rotating the knob may cause a nut to translate along an internal track of the knob, wherein the track includes a first stop and a second stop that are configured limit an extent of proximal and distal translation of the nut, thereby limiting the number of rotations of the knob and the catheter.

In this aspect, rotating the knob may cause rotation of a series of disks of a disk assembly, wherein the disks are rotationally linked to limit an extent of rotation of each of the disks of the disk assembly, thereby limiting the number of rotations of the knob and the catheter.

In this aspect, the rotation limiter may limit the number or rotations that the catheter rotates to no more than four rotations.

In this aspect, the guidewire lumen may run longitudinally along the nosecone but not along the elongate body.

In this aspect, the rotation limiter may provide tactile, audible, or tactile and audible feedback to a user rotating the knob.

In this aspect, the method may further comprise distally moving the driveshaft to cause the cutter to extend through a cutter window of the catheter, wherein the cutter window is between the nosecone and the elongate body.

In this aspect, the nosecone may be fixedly coupled to the elongate body at a fixed bend of the catheter.

In this aspect, a cutter window may be on a convex side of the fixed bend.

In this aspect, a cutter window may be distally located along the catheter with respect to the fixed bend.

In this aspect, the method may further comprise pivoting the nosecone relative to the elongate body.

In this aspect, the method may further comprise applying a force to the driveshaft to cause a shapeable section of the elongate body to take on a pre-determined shape.

In this aspect, the shapeable section may include a frame having at least one backbone.

The features, components, methods and apparatuses described herein may be used with and/or modify one or more of methods and apparatuses, and in particular the atherectomy devices, described in International Application No. PCT/US2020/056072, filed Oct. 16, 2020, entitled “ATHERECTOMY CATHETER WITH SHAPEABLE DISTAL TIP,” published as International Patent Publication No. WO 2021/076957; International Application No. PCT/US2017/040431, filed on Jun. 30, 2017, entitled “ATHERECTOMY CATHETER WITH SHAPEABLE DISTAL TIP,” published as International Patent Publication No. WO 2018/006041; and International Application No. PCT/US2019/028415, filed on Apr. 19, 2019, entitled “OCCLUSION-CROSSING DEVICES,” published as International Patent Publication No. WO 2019/204797, each of which is incorporated herein by reference herein in its entirety.

These and other aspects and advantages are described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows an atherectomy catheter having a fixed jog. FIG. 1B shows the atherectomy catheter of FIG. 1A in a vessel.

FIG. 2 shows a drawing of the atherectomy catheter of FIG. 1A with relative angles and dimensions.

FIG. 3A shows a variation of a distal end of an atherectomy catheter that includes a user-activated curved portion with stiffening members that cause the catheter to deform to a predetermined curved configuration when activated. FIG. 3B is a schematic showing the stiffening members of the atherectomy catheter of FIG. 3A.

FIGS. 4A and 4B show another embodiment of an atherectomy catheter with a distal curved portion.

FIG. 5A is a top view of a user-activated curved portion of an atherectomy catheter.

FIGS. 5B and 5C are perspective views of the curved portion of FIG. 5A. FIG. 5D is a flattened view of the curved portion of FIG. 5A.

FIG. 6A shows an atherectomy catheter including another embodiment of a user-activated curved portion. FIG. 6B shows the frame of the curved portion of FIG. 6A including annular and longitudinal spines. FIG. 6C shows a side view of the spine of FIG. 6B. FIG. 6D shows a cross-sectional view of the spine of FIG. 6B. FIG. 6E is a flattened view of the spine of FIG. 6B.

FIG. 7A shows a portion of an atherectomy catheter including a fixed jog section and a flexible section. FIG. 7B shows a flattened view of the curved portion of the catheter of FIG. 7A.

FIG. 8A shows an exemplary flexible nosecone for use with an atherectomy catheter.

FIG. 8B shows a flattened view of a portion of the nosecone of FIG. 8A.

FIG. 9A shows an atherectomy catheter including another embodiment of a user-activated curved portion. The catheter in FIG. 9A is in a straightened configuration. FIG. 9B shows the atherectomy catheter of FIG. 9A in a bent or activated configuration. FIG. 9C is a bottom view of a frame that forms the curved portion of the catheter of FIG. 9A in a straightened configuration. FIG. 9D is a top view of the frame that forms the curved portion of the catheter of FIG. 9A in a straightened configuration. FIG. 9E is a perspective view of the frame that forms the curved portion of the catheter of FIG. 9A in a bent or activated configuration. FIG. 9F is another perspective view of the frame that forms the curved portion of the catheter of FIG. 9A in a bent or activated configuration.

FIG. 10A shows a close-up partial section view of a catheter having a bushing configured to direct movement of a cutter through a cutting window. FIGS. 10B, 10C and 10D show close-up partial section views of the catheter of FIG. 10A as the cutter is progressively moved through the cutting window.

FIGS. 11A, 11B and 11C show a side view of a catheter having a flexible section configured to take on a curved shape.

FIG. 12 shows a side view of another catheter having a flexible section configured to take on a curved shape.

FIG. 13A shows a side view of a catheter having a fixed bend. FIG. 13B shows a close-up view of the fixed bend of the catheter of FIG. 13A. FIG. 13C shows a close-up partial section view of the catheter of FIG. 13A illustrating a cutter in a passive mode. FIG. 13D shows a close-up partial section view of the catheter of FIG. 13A illustrating a cutter in an active mode. FIG. 13E shows a close-up partial section view of the catheter of FIG. 13A illustrating a cutter between the passive mode and the active mode.

FIG. 14A shows a perspective view of an example cutter and bushing for a catheter having a fixed bend. FIG. 14B shows a section view of the cutter and bushing of FIG. 14A. FIG. 14C shows a perspective view of a proximal side of the bushing shown in FIG. 14A. FIG. 14D shows a perspective view of a distal side of the bushing shown in FIG. 14A. FIG. 14E shows a section view of the bushing shown in FIG. 14A. FIG. 14F shows another section view of the bushing shown in FIG. 14A. FIG. 14G shows a section view of the cutter and bushing of FIG. 14A illustrating a detent for retaining the cutter in a passive mode.

FIGS. 15A-15C show section views of a portion of a handle having a slider lock for locking an axial position of the driveshaft with respect to the catheter: FIG. 15A shows the slider lock engaged in a distal position; FIG. 15B shows the slider lock unengaged in the distal position; and FIG. 15C shows the slider lock engaged in a proximal position.

FIGS. 16A-16C show various views of an exemplary frame of a catheter body that is configured for unidirectional bending toward a backbone of the frame: FIG. 16A shows a side of the frame having a backbone; FIG. 16B shows a side of the frame opposite the backbone and having bend-control features; and FIG. 16C shows a further close-up view of bend-control features of the frame.

FIGS. 17A-17E show an exemplary frame configured for bidirectional bending: FIG. 17A shows a close-up view of bend-control features of the frame; FIG. 17B shows a side of the frame opposite the backbone as the frame is bending toward the backbone; FIG. 17C shows a side of the frame having the backbone as the frame is bending toward the backbone; FIG. 17D shows a side of the frame having the backbone as the frame is bending away from the backbone; and FIG. 17E shows a side of the frame opposite the backbone as the frame is bending away from the backbone.

FIG. 18 shows a closeup view of an exemplary frame of a catheter body that is configured for unidirectional bending away from a backbone of the frame.

FIGS. 19A and 19B show an exemplary frame that is configured to bend to an s-shape configuration: FIG. 19A shows a side view of the frame in a bent s-shaped configuration; and FIG. 19B shows a closeup side view of the frame in a neutral (straight) configuration.

FIG. 20 shows an exemplary frame that is configured to bend to U-shape configuration.

FIGS. 21A-21D show an exemplary frame 2133 that is configured to twist into a spiral shape up to 180 degrees: FIG. 21A shows a perspective view of the frame in a neutral (straight) configuration; FIG. 21B shows a first side view of the frame in a bent (spiral) configuration; FIG. 21C shows a second side view of the frame in a bent (spiral) configuration; and FIG. 21D shows a front perspective view of the frame in a bent (spiral) configuration.

FIGS. 22A-22C show an exemplary frame 2133 that is configured to twist into a spiral shape up to 270 degrees: FIG. 22A shows a first side view of the frame in a bent (spiral) configuration; FIG. 22B shows a second side view of the frame in a bent (spiral) configuration; and FIG. 22C shows a front perspective view of the frame in a bent (spiral) configuration.

FIGS. 23A-23C show an exemplary frame 2133 that is configured to twist into a spiral shape up to 360 degrees: FIG. 23A shows a first side view of the frame in a bent (spiral) configuration; FIG. 23B shows a second side view of the frame in a bent (spiral) configuration; and FIG. 23C shows a front perspective view of the frame in a bent (spiral) configuration.

FIG. 24 shows a side view of distal portion of an exemplary atherectomy catheter device, showing a guidewire lumen.

FIGS. 25A-25C show an exemplary rotation limiter, which may be part of a handle at a proximal end of the catheter device: FIG. 25A shows a side perspective view of the rotation limiter in an assembled state; FIG. 25B shows the rotation limiter in a disassembled state; and FIG. 25C shows a back view of a knob of the rotation limiter.

FIGS. 26A-26D show another exemplary rotation limiter, in this case, having a disk assembly: FIG. 26A shows a side partially sectioned view of the rotation limiter assembled in a handle; FIG. 26B shows a closeup side view of the disk assembly; FIG. 26C shows a closeup perspective view of a disk showing a tooth as part of a feedback feature; and FIG. 26D shows a closeup perspective view of a gear track configured to engage the teeth of the disk assembly to provide user feedback.

### DETAILED DESCRIPTION

Described herein is an atherectomy catheter having an elongate body with a curved distal portion, a nosecone and a rotatable annular cutter. The curved portion (which can otherwise be called a bent/bendable portion, shaped/shapable portion, or jog mechanism) can advantageously be used to push the cutter up against the vessel wall to enhance the efficiency of cutting.

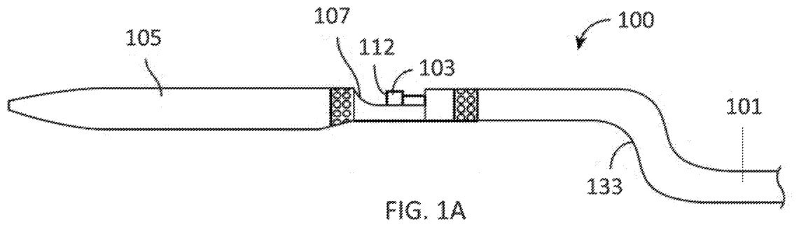

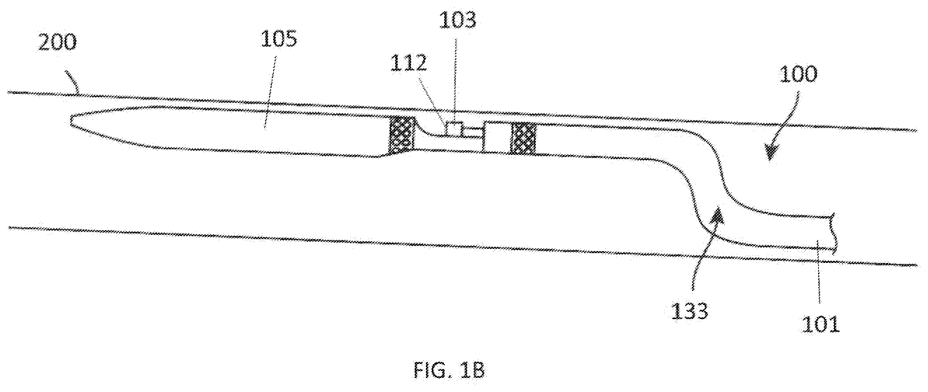

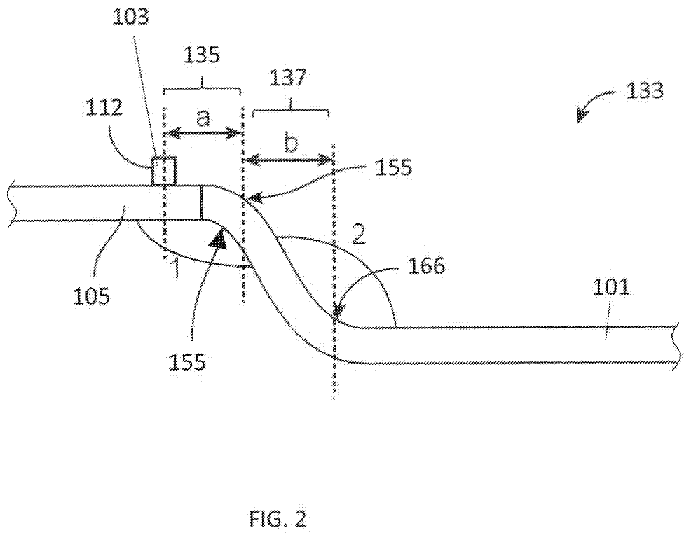

FIGS. 1A and 1B show an exemplary atherectomy catheter 100 having a curved portion along the elongate catheter body. Referring to FIGS. 1A-2, the atherectomy catheter 100 can include a catheter body 101 with a curved portion 133, a rotatable annular cutter 103 at a distal end of the catheter body 101, and a nosecone 105 at a distal end of the catheter body 101. The nosecone 105 can include a cutting window 107 configured to allow the cutter 103 to cut therethrough. The catheter 101 can further include a curved portion 133 in the catheter body 101 to radially push the cutter 103 against the vessel wall.

The curved portion 133 can be a fixed jog (i.e., have a pre-set shape). Further, the curved portion can be curved or bent such that the cutting window 107 is on the radially outermost portion of the curved portion 133 (thereby allowing the cutting window 107 to be urged against a vessel wall in use). In one embodiment, the curved portion 133 can be pre-formed, for example, by using pre-deflected shaped-set nitinol ribbon segments embedded in the outer shaft. The curved portion 133 can have a shape that advantageously positions the cutter 103 with respect to the vessel wall for cutting. In some cases, the curved portion 133 can have two inflection points 155, 166 of opposite curvature (i.e., one curving up and the other curving down) so as to form an approximate “s” shape. In one embodiment, the s-shape can be configured such that a distal end of the catheter body 101 is offset from, but substantially parallel to, a proximal end of the catheter body 101. In other embodiments, the distal end and proximal ends of the catheter body 101 can be at a slight angle to one another so as to control the angle of cutter engagement with the vessel wall.

Thus, as shown in FIG. 2, the “s-shape” of the curved portion 133 can include a proximal section 137 have a length b that extends from the center of the distal inflection point 155 to the center of the proximal inflection point 166. Further, the curved portion 133 can include a distal section 135 having a length a that extends from the cutting edge 112 to the center of the distal inflection point 155. Further, there can be distal angle 1 at the distal end of the “s-shape” and a proximal angle 2 at the proximal end of the “s-shape.” These lengths (a, b) and angles (1, 2) can be tuned to achieve the desired jog or offset in order to obtain optimum apposition to tissue walls. For example, the length a can be shorter than the length b to ensure that the cutter is as close to the angle 1 as possible, thereby providing better apposition of the cutter 303. The angles 1 and 2 can be between 120 and 180 degrees, such as between 140 and 160 degrees. In one example, the length a is between 5 and 10 mm, the length b is between 10 and 15 mm, the angle/is 140 degrees and angle 2 is 160 degrees for a catheter configured to be used in a vessel having a 2.5-4 mm diameter.

The curved portion 133 can advantageously radially push the distal end of the catheter against a vessel wall 200, thereby enabling optimized cutting and/or imaging of the vessel as shown in FIG. 1B.

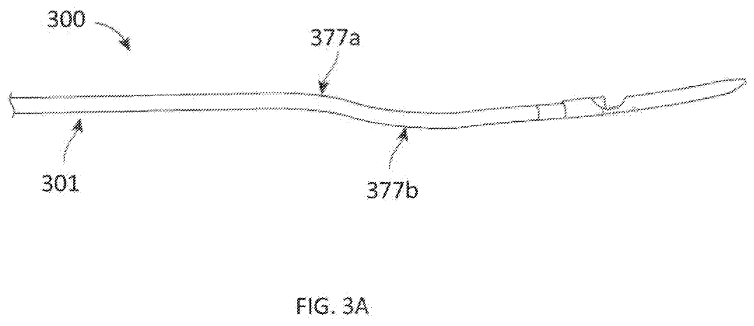

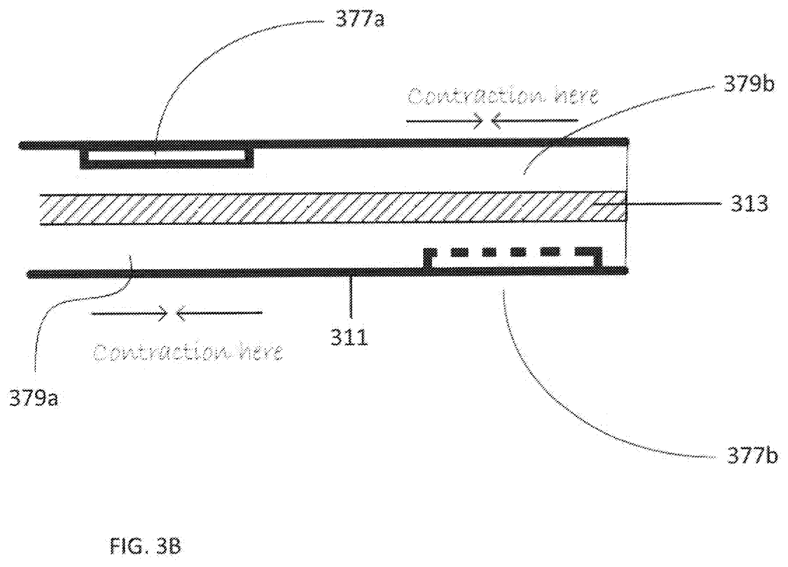

FIGS. 3A-3B show another embodiment of an exemplary catheter 300 that includes a curved portion 333 in the catheter body that urges the atherectomy cutter against the vessel wall. The curved portion 333 can have similar dimensions and features as curved portion 133. In contrast to the fixed jog curved portion 133 of catheter 100, however, the curved portion 333 can be a user-activated jog. Thus, referring to FIGS. 3A and 3B, the catheter 300, can be deflected into a curved portion 333 by tensile and compressive interaction between an inner shaft 313 (which can be a drive shaft for a cutter) and outer shaft 311 that are fixed together at the distal end but free to move relative to one another at the proximal end. The outer shaft 311 can include stiffening members 377a,b, such as nitinol or stainless steel, stiffening members, configured to bias the deflection to a set shape. As shown in FIG. 3B, there can be two stiffening members 377a, 377b that can be axially aligned with the outer shaft 311 and axially and radially offset from one another. As a result, when compression is applied on the outer shaft 311 (such as by pulling on the inner shaft 313 or a separate pullwire or shaft), the portions 379a,b of the outer shaft opposite to the stiffening members 377a,b will contract. The contraction of the two portions 379a, 379b will result in an s-shape similar to the catheter 100 shown in FIG. 1. As a result, the catheter will deflect into jog or s-shaped configuration where the distal end of the shaft is offset and parallel to the main shaft body. It is to be understood that other numbers and arrangements of stiffening members are possible, as are other resulting jog shapes.

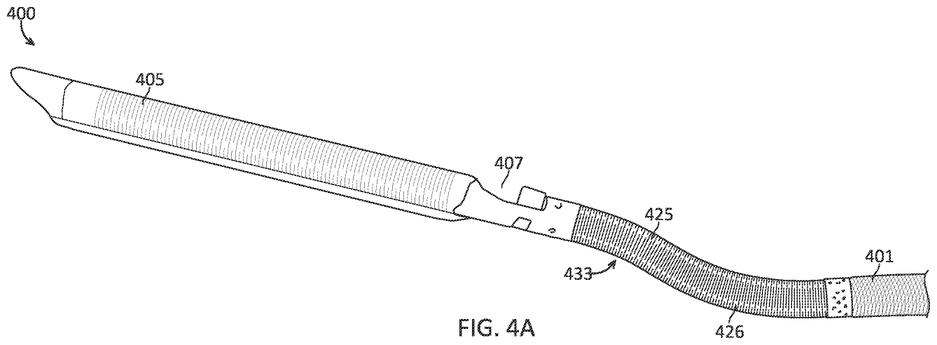

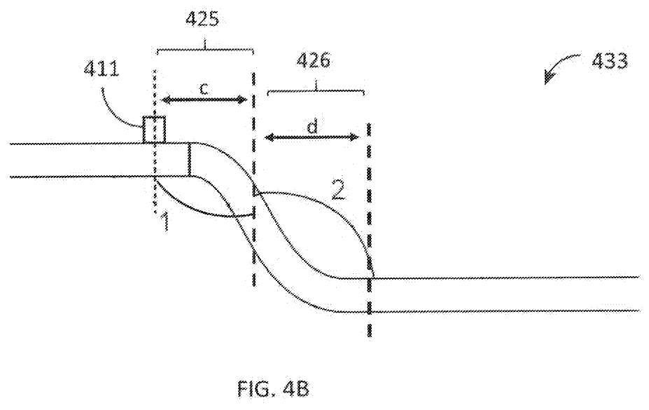

Another embodiment of an atherectomy catheter 400 including a user-activated curved portion 433 is shown in FIGS. 4A-4B. The atherectomy catheter 400 includes an elongate body 401, a nosecone 405 attached thereto, and a cutting window 407 configured to expose an annular cutter 411 therethrough. Moreover, the catheter 400 includes a curved portion 433. The curved portion 433 includes curved sections 425, 426 of opposite curvatures (i.e., one curving up and the other curving down) so as to form an approximate s-shape. In one embodiment, the s-shape can be configured such that the distal end of the catheter body 401 and/or the nosecone 405 is offset from, but substantially parallel to, a proximal end of the catheter body 401. In another embodiment, the distal end of the elongate body 401 and/or the nosecone 405 forms an angle relative to a proximal end of the catheter body 401.

Thus, as shown in FIG. 4B, the “s-shape” of the jog 433 can have a proximal curved section 426 and a distal curved section 425 having a length c. Further, there can be distal angle 1 at the distal end of the “s-shape” and a proximal angle 2 at the proximal end of the “s-shape.” The lengths (c, d) and angles (1, 2) of the jog 433 can be tuned to achieve the desired jog or offset in order to obtain optimum apposition to tissue walls. For example, the angles 1 and 2 can be between 120 and 175 degrees, such as between 140 and 160 degrees. Further, in some embodiments, the length d of the proximal section 426 is greater than the length c of the distal section 425. In one example, the length c is 5 mm, the length d is 8 mm, and the angles 1 and 2 are 150 degrees for a catheter configured to be used in a vessel having a 2.5-4 mm diameter. The curved portion 433 can be a configured to adopt the s-shape during use of the catheter, as described above with respect to curved portion 333.

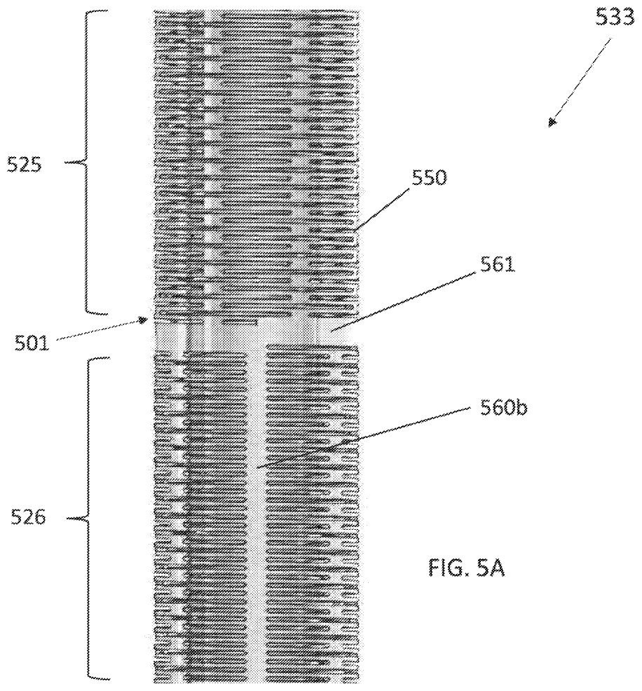

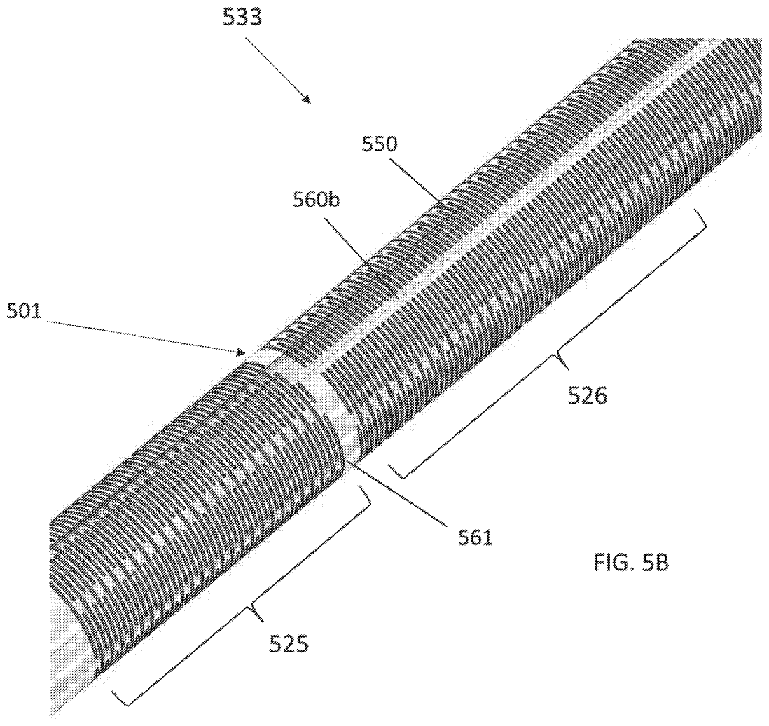

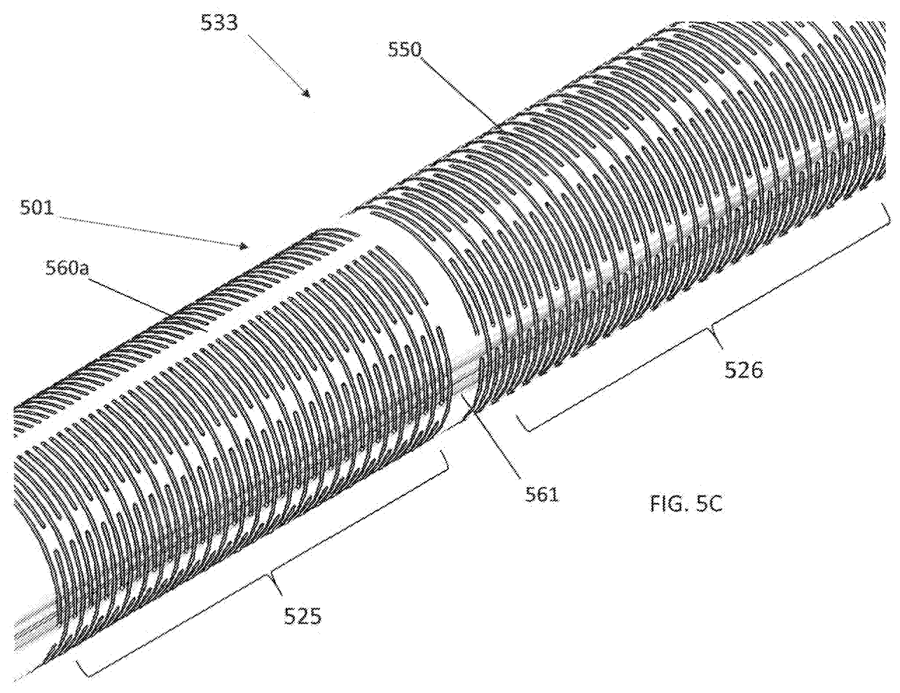

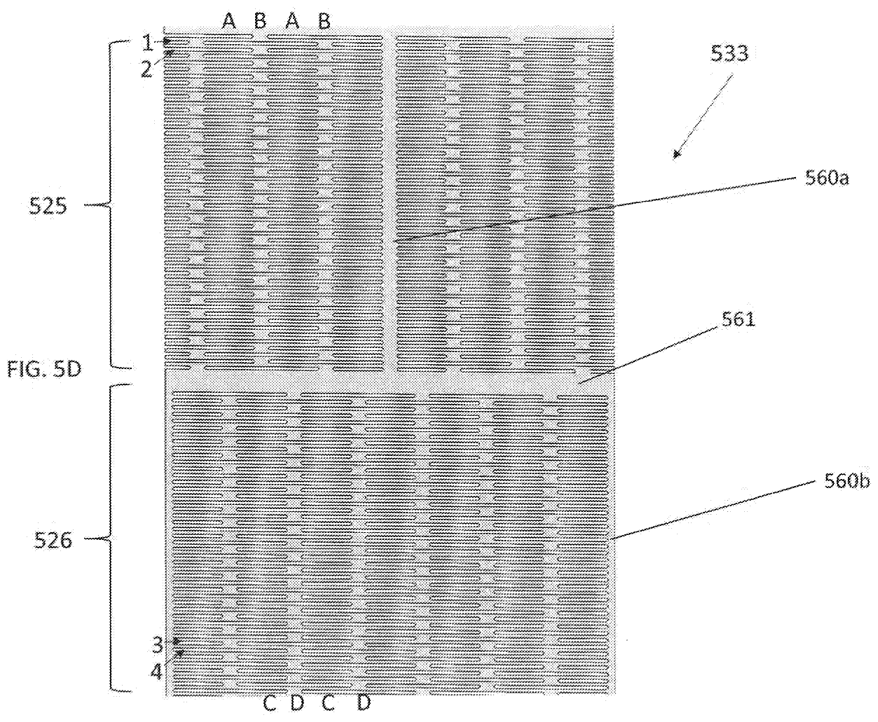

An exemplary user-activated curved portion 533 (e.g., for use as curved portion 433) is shown in FIGS. 5A-5D. The curved portion 533 can include a frame (e.g., made of Nitinol or stainless steel) including a series of circumferential slits 550 (e.g., laser cuts) that are patterned along the circumference of the elongate body 501 within the curved sections 525, 526. The frame of the curved sections 525, 526 can also include a longitudinal spine 560a,b (also referred to herein as a backbone) extending therethrough. The longitudinal spines 560a,b can be positioned approximately 180 degrees away from one another (i.e., on opposites sides of the elongate body 501) and extend substantially parallel to the longitudinal central axis of the elongate body 501. The frame can further include a circumferential spine 561 separating the two curved sections 525, 526. Each spine 560a,b and 561 is formed of a substantially solid piece of material that does not include slits therein. In use, as the circumferential slits 550 compress and/or overlap with one another during bending, the longitudinal spines 560a,b form the backbone of the curved sections 525, 526. Further, in some embodiments, the frame can be laminated with a layer thereover and/or under, such as a thin polymer layer, such as Tecothane. In other embodiments, the frame is not laminated to provide for greater flexibility.

Referring to FIG. 5D, the slits 550 can be arranged in a pattern that is configured to provide flexibility while maintaining structural integrity of the elongate body. Thus, the majority of the slits 550 can have the same length, but be offset from one another. For example, the slits in distal section 525 can be arranged in rows (1,2) and columns (A, B). Each slit 550 (except the shorter slits bordering the spine 560a) can have a length equivalent to the width of columns A+B+A. Further, the slits can be offset from one another by a distance of A+B. Thus, each column A can include slits from every row 1,2 while column B can include alternating slits (from either row 1 or 2). Column B thus provides structural integrity to the slitted portion of the device. The slits in section 526 can be similarly arranged, but can be offset such that each column C (with slits from every row 3,4) is aligned with the central axis of each column D (with slits from row 3 or 4). The offset helps provide stability to the catheter as it bends.

In some examples, pushing or pulling on a shaft of the catheter, such as the cutter drive shaft, a pullshaft, or a pullwire can activate the curved portion 533. That is, as the shaft is pulled back proximally, it can place compression on the outer elongate body 501, causing the slits 550 to compress and/or move over one another while the spines 560a,b maintain their length. The resulting s-shape (see FIG. 4B) allows the cutter (just distal to spine 460a) to be pushed up against the vessel wall.

The slits 550 shown in FIGS. 5A-5D are of a repeated, symmetrical pattern. However, the pattern need not be symmetrical. In some embodiments, the slits can all have the same length. In other embodiments, some of the slits are longer than others. In one embodiment, the slits are .0016″ wide and .0575″ long with a .0035″ offset from the next row of slits.

Areas of the catheter body having a greater degree of slits will be more flexible than those having lesser degrees of slits. In one embodiment, the slits can extend all the way through the elongate catheter. In other instances, some of the slits may be deeper or shallower than others which also affects the flexibility of the corresponding region. In some variations of the curved portion, a range of deflection between the flexible segments may be achieved. This may be accomplished through different geometric patterns of slits, different spacing of the slits, frequency of the slits, size of the slits, and so forth. In some instances, the degree of stiffness may be adjusted by adding additional spines of various lengths in certain areas or adjusting the width of the spines.

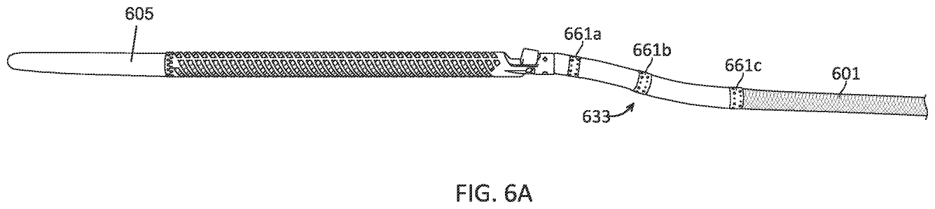

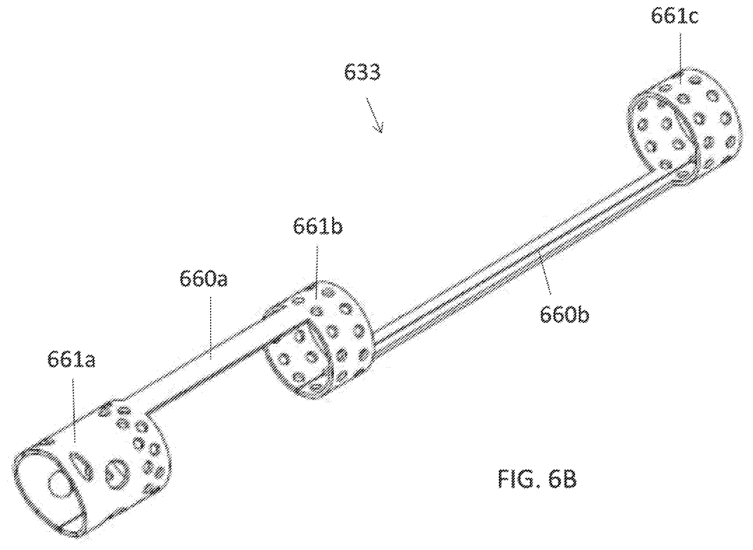

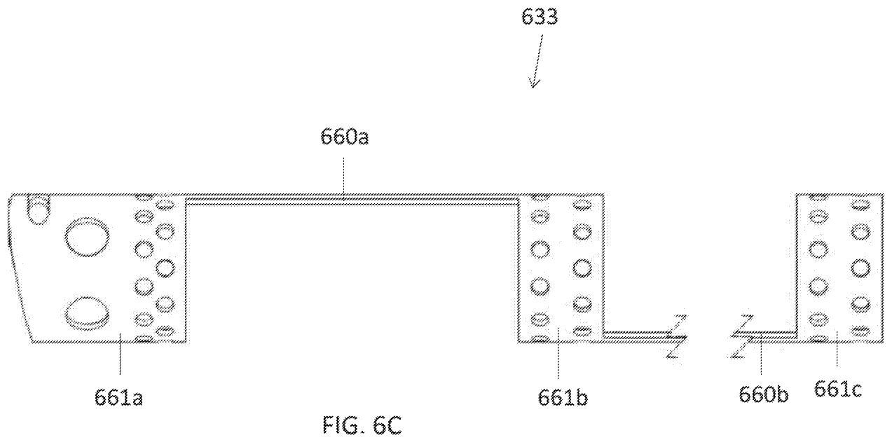

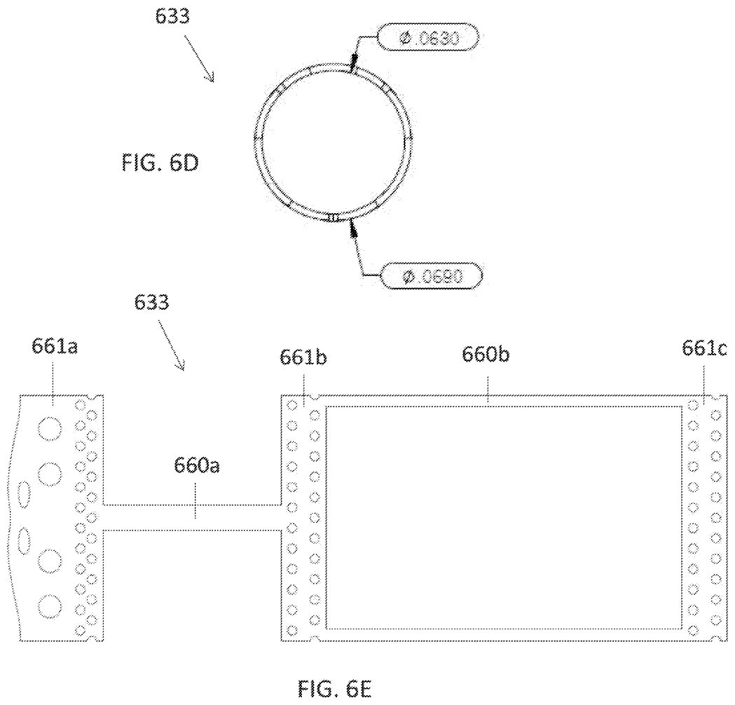

Referring to FIGS. 6A-6E, another exemplary curved portion 633 (e.g., for use as curved portion 433) is shown. The curved portion 633 includes a frame having three annular ring spines 661a,b,c connected together by two longitudinal spines 660a,b. The longitudinal spines 661a,b,c can be approximately 180° away from one another. In some embodiments, the distal ring 661a,bc can be beveled at the distal end, as shown in FIG. 6C, to allow for dropping or pivoting of the nosecone 605. Further, the space between the annular ring spines 661a,b,c and the longitudinal spines 660a,b can be open or cut-away (i.e., not include the frame material). In some embodiments, the frame can be laminated to the elongate body 601 with one or more thin polymer layers, such as Tecothane. The ring 661a,b,c can include holes therein for soldering or laminating the mechanism 633 to the elongate body of the catheter. In other embodiments, the frame can remain unlaminated to provide for greater flexibility. When compression is placed upon the mechanism 633, the mechanism 633 can bend away from each of the spines 660, forming an s-shape. For example, compression can be placed on the mechanism 633 by pulling the driveshaft is pulled proximally.

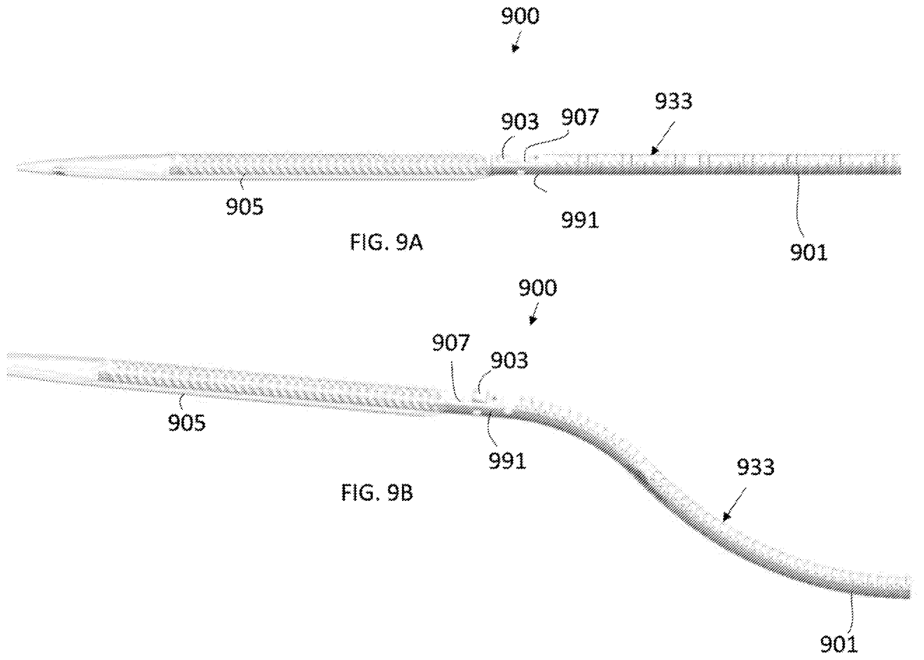

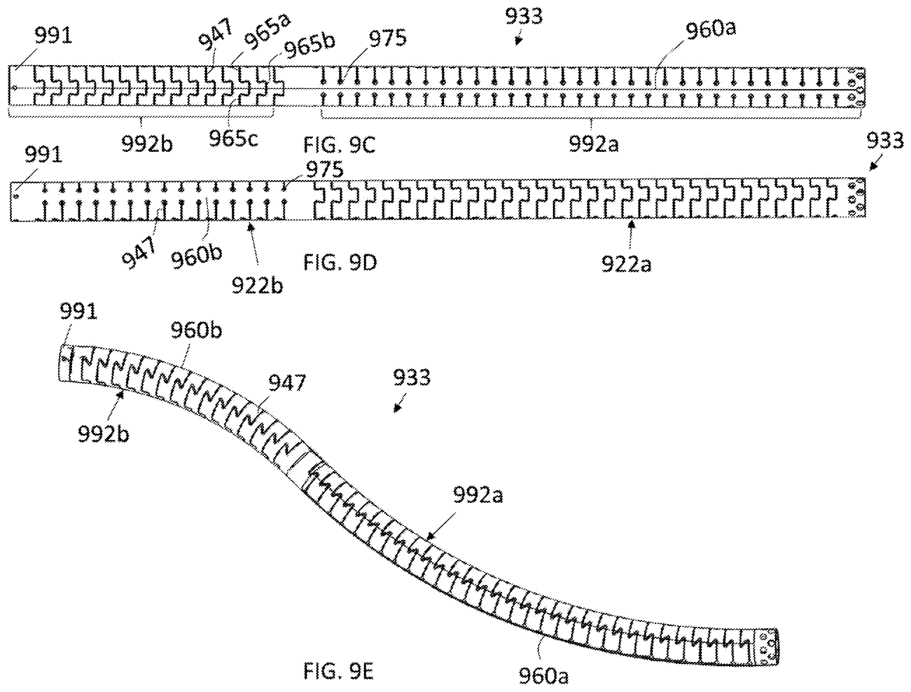

Referring to FIGS. 9A-9F, another exemplary atherectomy catheter 900 with a curved portion 933 is shown. The atherectomy catheter 900, like atherectomy catheter 100, can include a catheter body 901 with curved portion 933, a rotatable annular cutter 903 at a distal end of the catheter body 901, and a nosecone 905 at a distal end of the catheter body 901. The catheter body 901 and/or nosecone 905 can further include a cutting window 907 configured to allow the cutter 903 to cut therethrough. The catheter 900 can further include a driveshaft (not shown) attached to the cutter 903 and configured to rotate the cutter 903 when activated. In this embodiment, the nosecone 905 is not hinged relative to the elongate body 901. Rather, the bushing 991 of the catheter body 901 attaches directly to the proximal end of the nosecone 905. In some cases, not having a hinge may advantageously prevent the nosecone 905 from getting caught within the vessel. In this example, proximal or distal movement of the cutter 903 and driveshaft can activate the curved portion 933 to push the cutter 903 radially against the vessel well (e.g., outside the cutting window 907).

As shown in FIGS. 9C-9F, the curved portion 933 includes a tubular frame (also referred to herein as a scaffold) having a proximal section 992a and a distal section 992b. Each section 992a, 992b includes a longitudinal spine 960a,b. The longitudinal spines 960a,b are positioned approximately 180 degrees away from one another. Cuts 947 (e.g., laser cuts), also referred to herein as slits, extend circumferentially partially around the catheter body 901 and end at terminuses 975 on one side of the catheter body 901. In this example, the terminuses 975 are enlarged (e.g., shaped semi-circular holes), which may serve to relieve strain. The slits 947 have a jigsaw pattern that define tongue elements 965a,b,c (see FIG. 9C), which may control an extent to which the catheter body bends. In this example, the slits 947 in each section 992a, 992b are positioned opposite the side of the catheter body 901 having respective spines 960a,b. When compression is placed upon the mechanism 933, the mechanism 933 can bend into an s-shape with the proximal section 992a bending in a first direction and the distal section 992b bending in a second opposite direction. As shown in FIGS. 9E and 9F, during such bending, the spines 960a, 960b do not compress together and thereby form the outer diameter of each respective curve, while the cuts 947 compress together to form the inner diameter of each respective curve.

In some embodiments, the tongue elements 965a,b,c may have a tapered structure configured to dictate the amount of deflection of the curved portion 933 in both directions. For example, the tongue elements 965a,b,c may be configured to lock with respect to one another in the curved position, thereby keeping the curved portion 933 aligned and resistant to twisting when under torsion when in the curved or deflected position. This can also prevent the curved portion 933 from over-bending.

In some embodiments, the proximal section 992a can be longer than the distal section 992b. For example, the proximal section 992a can form 60-90%, such as 65%-70% of the length of the curved portion 933 while the distal section 992b can form 10%-40%, such as 30%-35% of the length of the curved portion 933. Having a longer distal section 992b than proximal section 992a can advantageously help ensure that the cutter 903 is forced against the vessel well during use without tipping back down towards the center of the vessel.

The curved portion 933 can be coupled to the outer shaft of the atherectomy catheter using any technique, such as welding, adhesive, fastener(s), or a combination thereof (e.g., via holes 907).

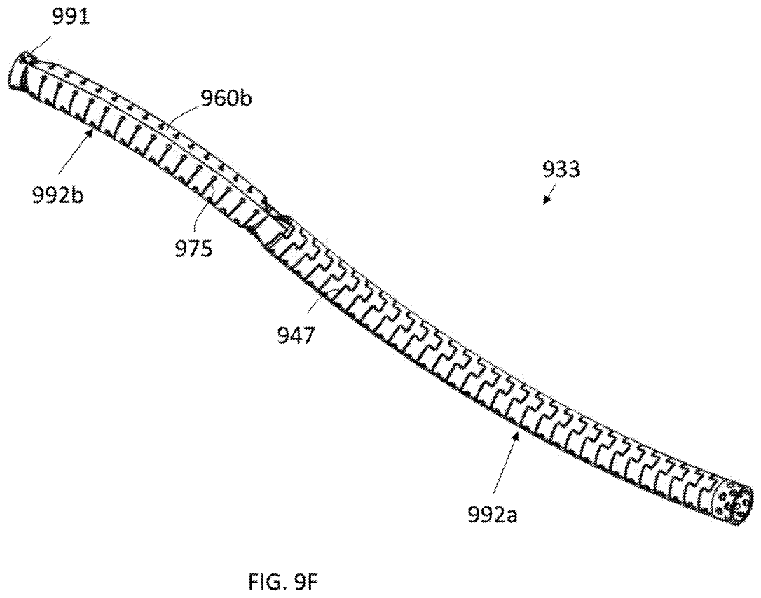

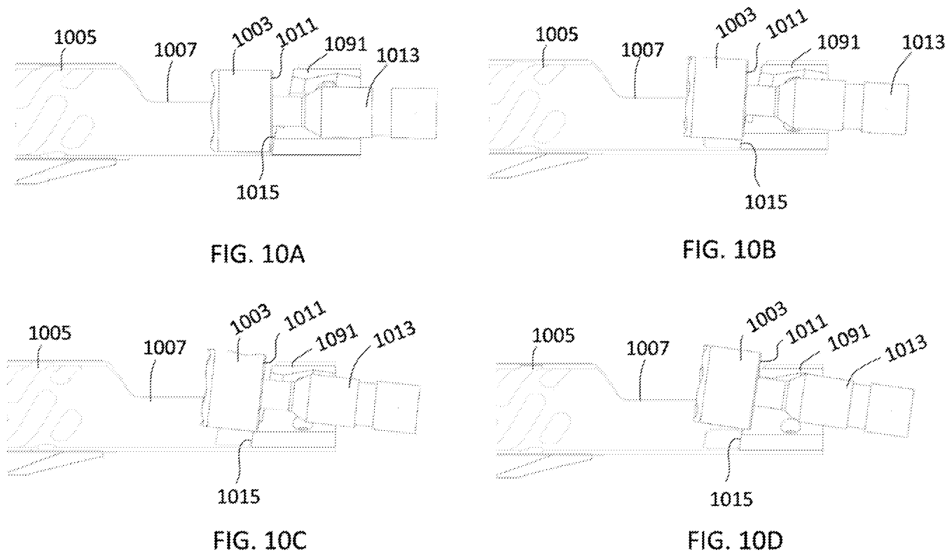

Referring to FIGS. 10A-10D, in any of the embodiments described herein, the cutter 1003 can include a proximal ledge 1011 configured to interact with the bushing 1091 on the curved portion 1033 when the driveshaft 1013 is pulled proximally. The curved portion 1033 can correspond to a flexible section of the catheter. In embodiments wherein the nosecone 1005 is not hinged, the interaction between the proximal ledge 1011 and the bushing 1091 can cause the cutter 1033 to move through the window 1007. For example, the distal edge 1015 of the bushing 1091 can be angled such that, as the drive shaft 1013 is pulled proximally, the ledge 1011 slides along the sloped distal edge 1015 (e.g., sloped with respect to an axis perpendicular to the longitudinal axis of the nosecone 1005) to move the cutter 1003 out of the cutter window 1007 (see the movement from FIGS. 10A-10D). That is, interaction between the ledge 1011 and the edge 1015 can cause at least a portion of the cutter to extend through (e.g., pop through) the window 1007 and tilt such that the cutter 1003 is at an angle relative to the nosecone 1005 and the elongate body (e.g., 901 in FIGS. 9A and 9B). In other words, movement of cutter 1033 proximally relative to the nosecone can cause at least a portion of the cutter to extend through the window and cause a longitudinal axis of the cutter 1033 to become non-parallel to a longitudinal axis of the nosecone 1005 and the elongate body. In some embodiments, the longitudinal axis of the cutter 1033 is at an angle ranging from about 1 degree and 30 degrees (e.g., 1°−30°, 5°-30°, 20°-30°, 1°-20°, or) 10°-20° relative to the longitudinal axes of the nosecone 1005 and elongate body when the cutter 1003 is fully deployed.

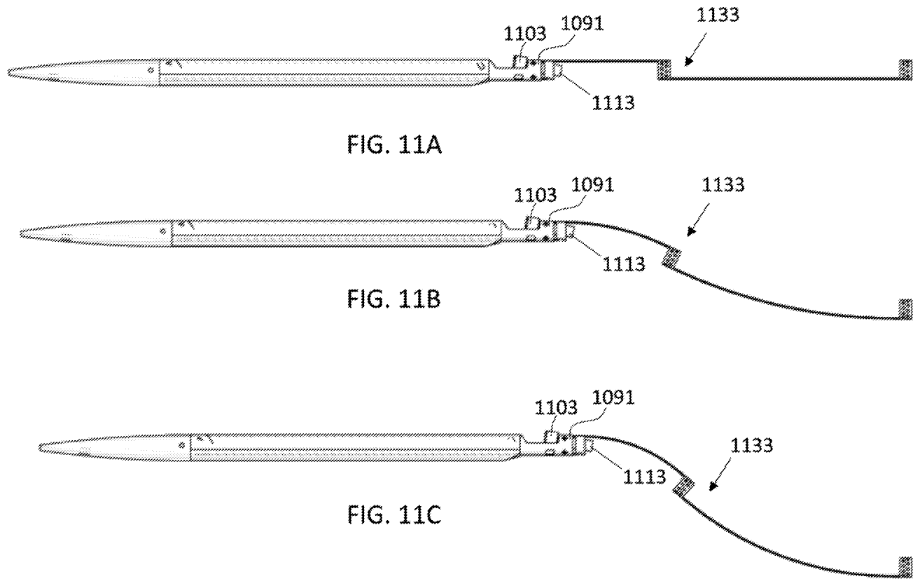

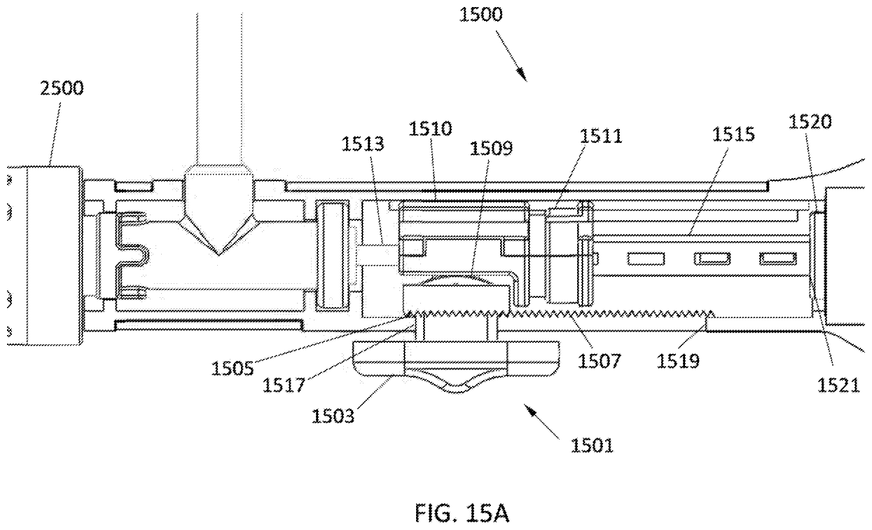

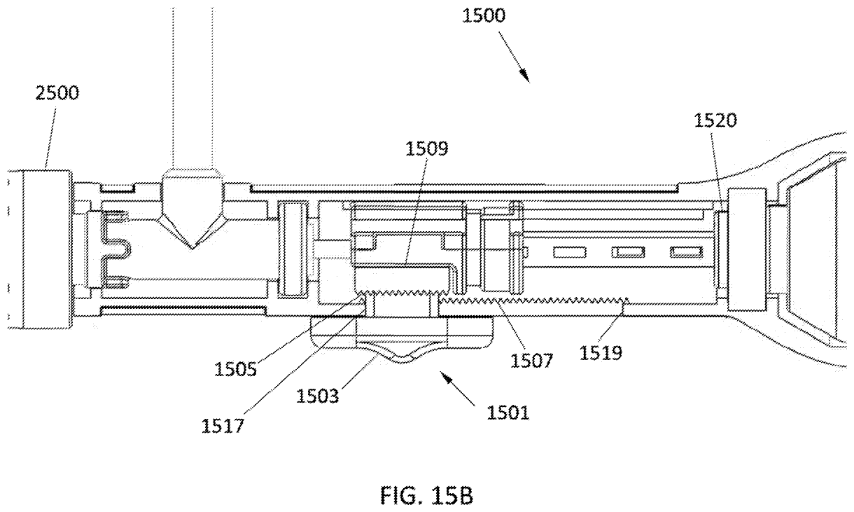

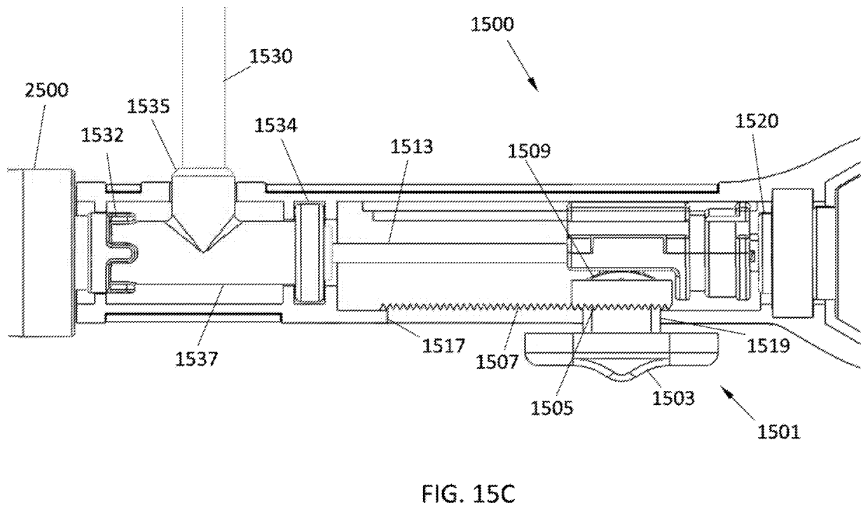

In some embodiments, the interaction between the proximal ledge and the bushing can additionally or alternatively cause the curved portion (also referred to as a flexible section) to assume its s-shape. For example, referring to FIGS. 11A-11C, pulling the driveshaft 1113 (shown cut off for clarity) proximally can cause the cutter 1103 to engage with the bushing 1191 to place compression on the curved portion 1033 and force the curved portion 1133 to bend (i.e., away from each of the longitudinal spines 1160a,b). In some embodiments, the amount of curvature can be incrementally and/or continuously adjustable by placing varying amounts of compression on the curved portion 1133 via the driveshaft. Likewise, pushing the driveshaft 1113 distally can straighten the curved 1133. A locking mechanism, such as a mechanism on the handle, can fix the curved portion 1133 at the desired amount of curvature. An example of a handle with locking mechanism is described below with reference to FIGS. 15A-15C.

In some embodiments, the cutter 1003 is pulled proximally by a first extent and/or at a first time to extend a portion of cutter 1003 through the window 1007 and tilt relative to the nosecone and elongate body (e.g., as shown in FIGS. 10A-10D), and pulled proximally by a second and/or at a second time to cause the flexible portion to bend and assume an s-shape to varying degrees depending on the amount of force applied to the driveshaft in the proximal direction (e.g., as shown in FIGS. 11A-11C).

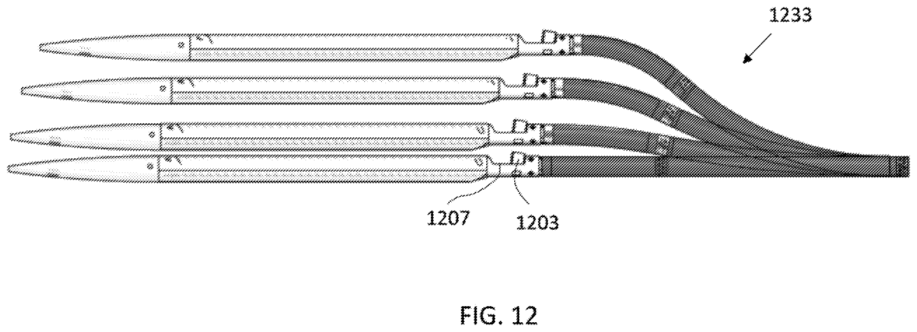

Referring to FIG. 12, in some embodiments, pulling the cutter 1203 proximally (e.g., via the driveshaft) can first cause the cutter 1203 to pop out of the cutter window 1207. Further proximal pulling on the cutter 1203 can then cause the curved portion 1233 to assume the desired s-shape (e.g., in a continuously adjustable manner). Having the cutter 1203 pop out of the window first can advantageously ensure that the cutter 1203 can be fully extended regardless of the assumed curvature. Pushing the cutter distally can cause the curved portion 1233 to straighten to a desired extent. Further distal pushing on the cutter 1203 can cause the cutter 1203 to retract within the window 1207.

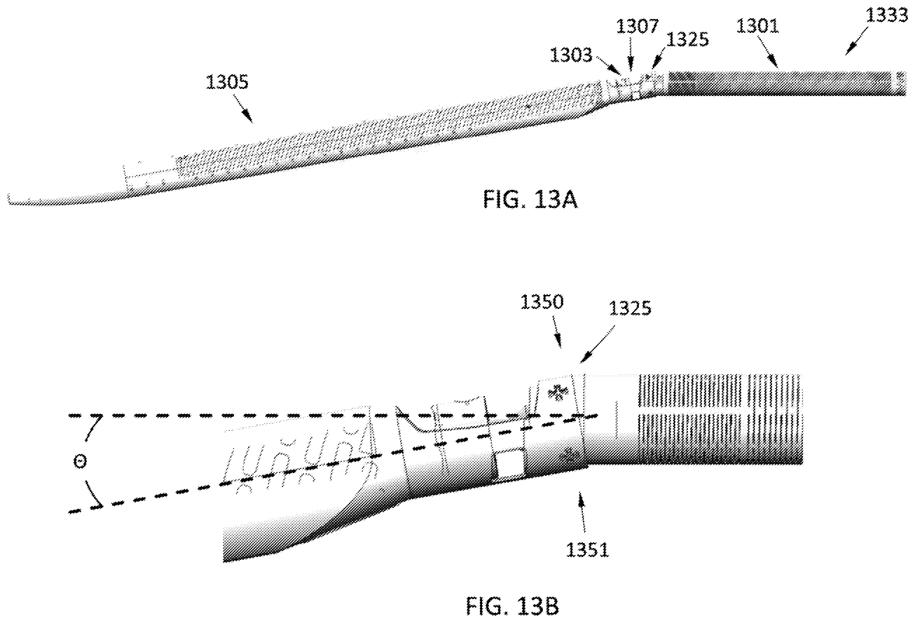

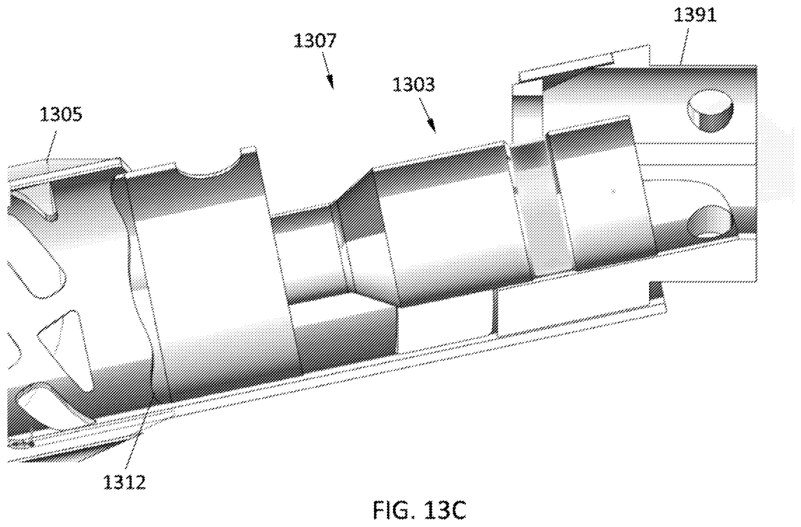

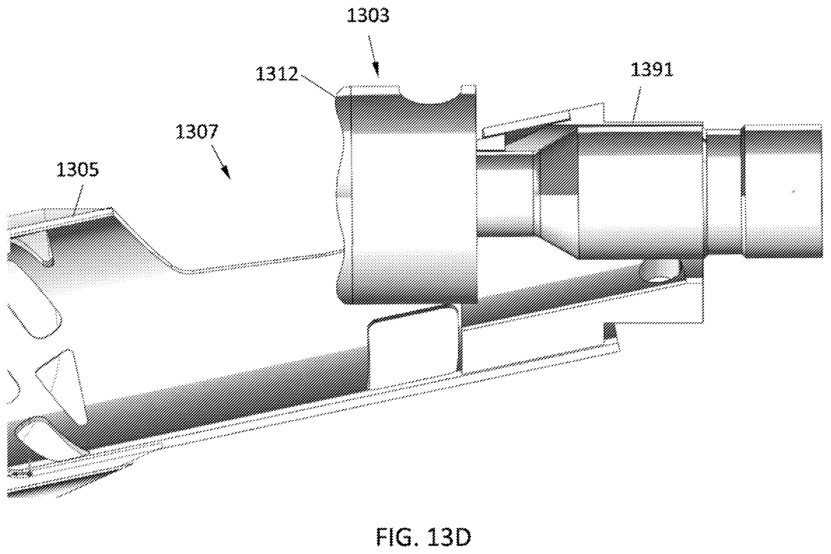

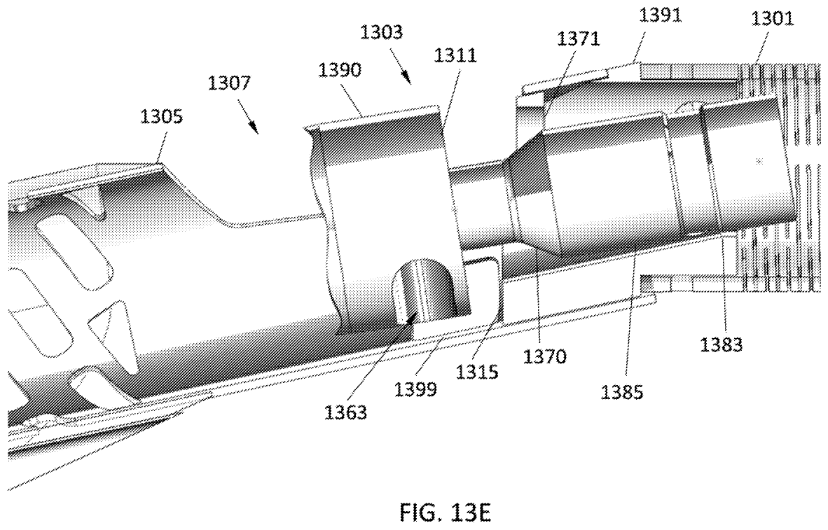

Referring to FIGS. 13A and 13B, the nosecone 1305 can be fixedly coupled to or integrally formed with the elongate body 1301 at a fixed angle θ relative to the elongate body. This fixed angle can form a fixed bend 1325 (also referred to as a fixed curve) in the catheter between the nosecone 1305 and the elongate body 1301. A cutting window 1307 can provide access to the lumen of the catheter where the cutter 1303 is housed. The cutting window 1307 can be located adjacent to or at the fixed bend 1325. In some embodiments, the cutting window 1307 is distally located along the catheter with respect to the bend. The cutter 1303 can be configured to extend through the cutting window 1307 upon translation of the cutter 1303 and driveshaft relative the catheter (i.e., nosecone and elongate body). For example, the driveshaft and cutter 1303 can be pulled to move the cutter 1303 proximally and extend the cutter 1303 though the cutting window. Likewise, the driveshaft and cutter 1303 can be pushed to move the cutter 1303 distally and retract the cutter 1303 into the catheter housing.

The cutting window 1307 can be on a convex side 1350 of the catheter formed by the bend (e.g., as opposed to a concave side 1351 of the catheter formed by the bend). This configuration can provide the rotating cutter 1303 better access to material outside of the catheter for cutting. The angle θ of the bend 1325 can vary. In some embodiments, the angle θ ranges from about 1 degree and 30 degrees (e.g., 1°-30°, 5°-30°, 20°-30°, 1°-20°, or) 10°-20°. Thus, in some embodiments, the angle of the bend 1325 at the convex side 1350 of the catheter may range from about 181° and 210° (e.g., 181°-210°, 186°-210°, 200°-210°, 186°-200°, or 190°-200°).

The elongate body 1301 can include a flexible section 1333 consistent with the flexible section as described above with reference to FIGS. 11A-11C and 12. For example, the flexible section 1333 can be incrementally and/or continuously adjustable to take on an s-shape to varying degrees depending on an amount of proximal movement of driveshaft relative to the nosecone 1305 and elongate body 1301. The flexible section 1333 can have a greater flexibility than the nosecone 1305, the bend 1325, and in some cases a remainder of the catheter.

FIGS. 13C-13E show close-up partial section views of the bend 1325. The cutter 1303 can be configured to transition between a passive mode, such as shown in FIG. 13C, and an active mode, such as shown in FIG. 13D. FIG. 13E shows the cutter 1303 between the passive mode (FIG. 13C) and active mode (FIG. 13D).

Referring to FIG. 13C, when the cutter 1303 is in passive mode, the cutting edge 1312 of the cutter 1303 can be distally located with respect to the window 1307 and housed within the nosecone 1305 such that the cutting edge 1312 of the cutter 1303 is fully protected and does not extend through the cutting window 1307. In the passive mode, the cutting edge 1312 may be prevented from cutting material outside of the catheter, thereby preventing the cutting edge 1312 from cutting tissue, for example, a vessel wall. The cutter 1303 may be placed in the passive mode, for example, as the catheter is being maneuvered through the vessel to arrive at a target location within the vessel (e.g., to remove material such as plaque) and/or being withdrawn from the vessel (e.g., after removal of the material from the vessel). In the passive mode, a longitudinal axis (e.g., axis of rotation) of the cutter 1303 can be substantially parallel to a longitudinal axis of the nosecone 1305. In the passive mode, the cutter 1303 can be pushed distally toward the nosecone 1305 to, for example, pack material (e.g., plaque) into the nosecone 1305.

Referring to FIG. 13D, when the cutter 1303 is in the active mode, the cutting edge 1312 of the cutter 1303 can extend through the cutting window 1307. The cutter 1303 (e.g., the cutting edge 1312 of the cutter 1303) can extend beyond the outer walls on the convex side of the catheter so that the cutter 1303 can efficiently access material outside of the catheter for cutting. For example, at least a portion of the cutter 1303 (e.g., at least a portion of the cutting edge 1312) can correspond to the most prominent feature or point along the convex side of the bend when the cutter 1303 is extended through the cutting window 1307 (e.g., fully extended in the active mode). In the active mode, a longitudinal axis (e.g., axis of rotation) of the cutter 1303 can be substantially parallel to a longitudinal axis of the elongate body (e.g., 1301, FIG. 13A). Since the cutter 1303 can be parallel to the elongate body, the cutter 1303 can be at the angle Q (FIG. 13B) relative to the nosecone 1305 when in the active mode.

FIG. 13E shows the cutter 1303 between the passive mode and the active mode. The cutter 1303 and driveshaft can be moved proximally (e.g., pulled away from the nosecone 1305) to transition the cutter 1303 from the passive mode to the active mode. Likewise, the cutter 1303 and driveshaft can be moved distally (e.g., pushed toward the nosecone 1305) to transition the cutter 1303 from the active mode to the passive mode. During transition between the passive and active modes, the cutter 1303 can be configured to interact with inner surfaces within the catheter to adjust the position and orientation of the cutter 1303 relative to the nosecone 1305 and the elongate body 1301.

For example, during transition from the passive mode to the active mode, the cutter 1303 (e.g., via the driveshaft) is pulled proximally to cause a proximal ledge 1311 (also referred to as a proximal face) of a head 1390 of the cutter 1303 to slide along a distal edge 1315 of a bushing 1391. This interaction causes the cutter 1303 to move radially outward with respect to a central axis of the elongate body 1301 and extend through the cutting window 1307 (e.g., pop out of the window). This interaction also causes the cutter 1303 to tilt such that a longitudinal axis of the cutter 1303 aligns with (e.g., becomes substantially parallel to) a longitudinal axis of the elongate body 1301.

During transition from the active mode to the passive mode, the cutter 1303 (e.g., via the driveshaft) is pushed distally to cause a slanted surface 1370 along the shaft 1385 of the cutter 1303 to slide along an internal edge 1371 of the bushing 1391 to cause the cutter 1303 to move radially inward with respect to a central axis of the elongate body 1301 and retract into the catheter. When the cutter 1303 is moved radially inward, the shaft 1385 of the cutter 1303 contacts an internal surface 1383 of the bushing 1391, causing the cutter 1303 to tilt such that the longitudinal axis of the cutter 1303 aligns with (e.g., becomes substantially parallel to) a longitudinal axis of the nosecone 1305.

The transitions between the passive and active modes can be continuous, where the cutter 1303 progressively translates, tilts and moves radially. The cutter 1303 and drive shaft can freely rotate while in the passive mode and the active mode. In some cases, the cutter 1303 can also freely rotate while transitioning between the passive mode and the active mode.

The cutter 1303 can be locked in either the passive or active modes using a locking mechanism of the handle. An example of a locking mechanism is described below with reference to FIGS. 15A-15C.

Any of the catheters described herein may include imaging capabilities such as described in International Application Nos. PCT/US2017/040431 (published as WO 2018/006041) and PCT/US2019/028415 (published as WO 2019/204797), each of which is incorporated herein by reference herein in its entirety. For example, the cutter 1303 can include a cavity 1363 for an imaging sensor within the catheter to send and/or receive image data as part of an imaging system. The cutter 1303 may be configured to collect imaging data while in the passive mode, the active mode and/or while transitioning between the active and passive modes. In some embodiments, the catheter includes one or more openings 1399 that act as an additional window and/or as a location marker(s) for the imaging sensor.

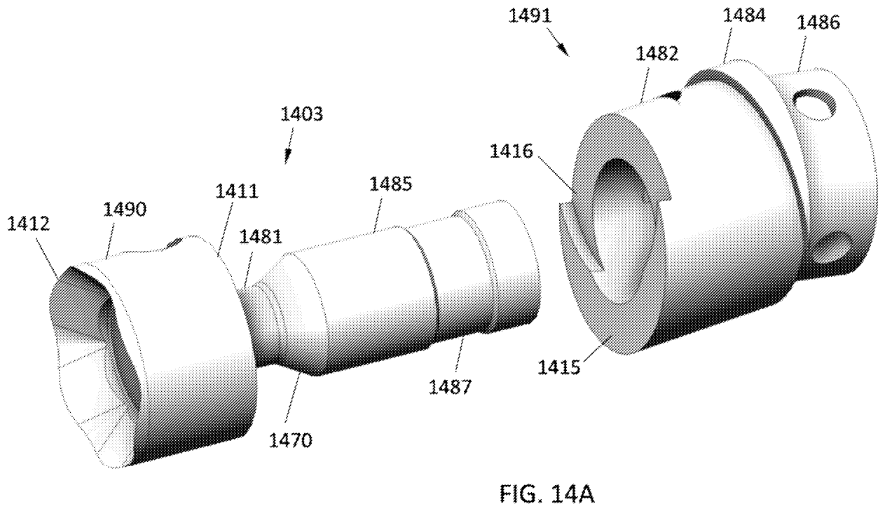

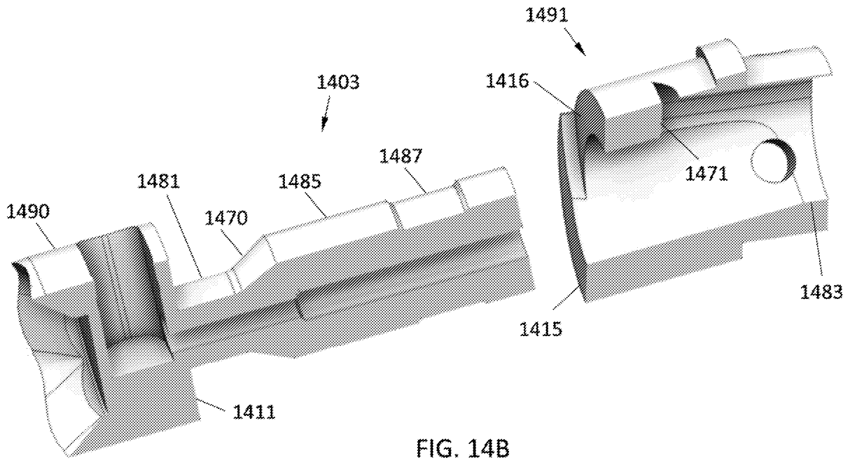

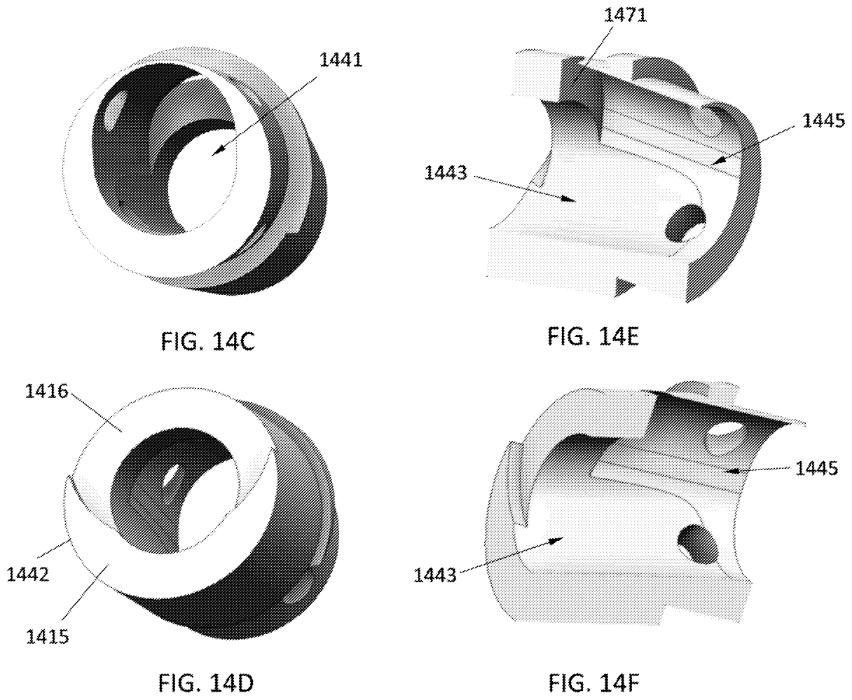

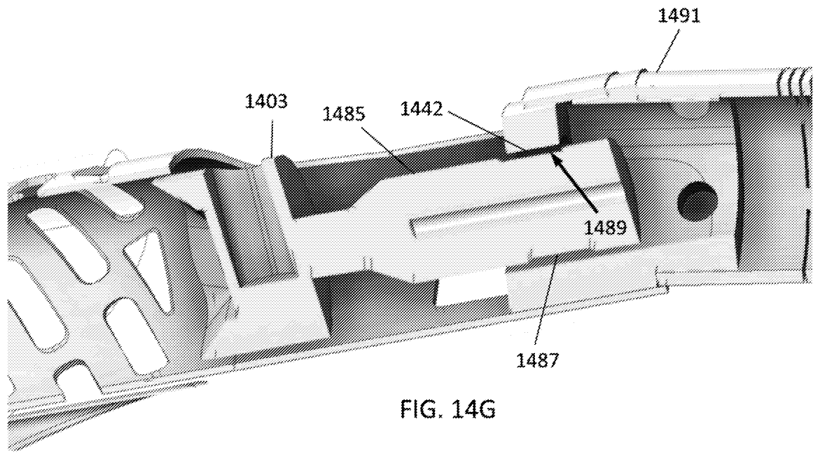

FIGS. 14A and 14B show perspective and section views of an example cutter 1403 and bushing 1491. The cutter 1403 can include a head 1490 at a distal end, a neck 1481, and a cylindrical shaft 1485 at a proximal end. The head 1490 can include an annular cutting edge 1412, which may scalloped in some embodiments. The neck 1481 can have a smaller diameter than the head 1490 and the proximal shaft 1485 to provide a clearance for the cutter 1403 when rotating in the active mode. In some embodiments, the shaft 1485 can include an annular groove 1487 that cooperates with the bushing 1491 to function as a detent (described in detail below). The bushing 1491 can be fixedly coupled to and be positioned between the distal nosecone (e.g., 1305, FIG. 13A) to the proximal elongate body (e.g., 1301, FIG. 13A). In some cases, the bushing 1491 is welded to the nosecone and/or the elongate body. The bushing 1491 can include a first portion 1482 that is substantially parallel the nosecone, and a second portion 1486 that is substantially parallel the elongate body. An intervening portion 1484 can be between the first portion 1482 and the second portion 1486.

As described above, features of the bushing 1491 can interact with the cutter 1403 to control movement of the cutter 1403 between active and passive modes. When the cutter 1403 is pulled proximally (e.g., from the passive mode to the active mode), the proximal ledge 1411 (also referred to as a proximal face) of the head 1490 of the cutter 1403 can be configured to slide along a distal edge 1415 of the bushing 1491. This interaction causes the cutter 1403 to move radially outward and extend through the cutting window. This cutter 1403 becomes positioned within a notch 1416 (also referred to as a seal or indentation) of the distal face of the bushing 1491, which provides a space for the proximal ledge 1411 of the cutter 1403 to rotate in the active mode. As shown in FIG. 14A, the distal edge 1415 may form a crescent-shaped step in accordance with the cylindrical head 1490 of the cutter 1403.

Referring to FIG. 14B, the bushing 1491 can include an internal edge 1471 that is configured to slide along an angled surface 1470 the cutter 1403 when the cutter is pushed distally (e.g., from the active mode into the passive mode), causing the cutter 1403 move radially inward. Additionally, an internal surface 1483 of the bushing 1491 contacts the shaft 1485 of the cutter 1403 to cause the cutter 1403 to tilt and move in alignment with and retract within the nosecone, as described above.

FIGS. 14C-14F shows alternate views of the bushing 1491, illustrating a proximal side (FIG. 14C), a distal side (FIG. 14D), and section views (FIGS. 14E and 14F) of the bushing. A proximal opening 1441 of the bushing can have an oblong shape to provide clearance for the shaft of the cutter when transitioning between the active and passive modes. The internal surfaces of the bushing can form a first channel 1443 for the cutter while in the passive mode and a second channel 1445 for the cutter while in the active mode. The first channel 1443 and the second channel 1445 can be configured to retain the cutter at different angles based on whether the cutter is in the passive mode or active mode. The first channel 1443 can retain the cutter in alignment with (e.g., parallel to) the nosecone, and the second channel 1445 can retain the cutter in alignment with (e.g., parallel to) elongate body. The first distal surface 1415 can be on a protruding lip 1442 on the distal end of the bushing. As described above, the first distal surface 1415 of the bushing can slide along an angled surface a proximal face of the head of the cutter to urge the cutter from the passive mode to the active mode. Also as described above, a surface 1471 of the bushing can slide along an angled surface along the shaft of the cutter to urge the cutter from the active mode to the passive mode.

As described above, the cutter 1403 can be retained in the passive mode by detent mechanism. FIG. 14G shows an annular groove 1487 on the shaft 1485 of the cutter 1403, which provides a clearance 1489 (also referred to as a gap) between the cutter and a protruding surface 1442 of the bushing. The clearance 1489 allows the cutter to rotate more freely when the annular groove 1487 is aligned with the internal surface 1442 and the cutting edge of the cutter is housed within the catheter in the passive mode. This configuration can act as a detent to retain the cutter 1403 in the passive mode. For instance, when the cutter is pushed more distally toward the nosecone (e.g., during packing of material into the nosecone) or when the cutter 1403 is pulled more proximally during transition to the active mode, the groove 1487 is not aligned with the internal surface 1442. This causes portions of the shaft 1485 on either side of the groove 1487 to contact the internal surface 1442 of the bushing 1491, thereby increasing a drag (friction) between the cutter 1403 and the bushing 1491. A threshold translational force applied to the cutter 1403 may be required, either in the distal or proximal direction, to release the detent retaining the cutter 1403 in the passive mode.

In some embodiments, any of the atherectomy devices described herein may not include imaging capability.

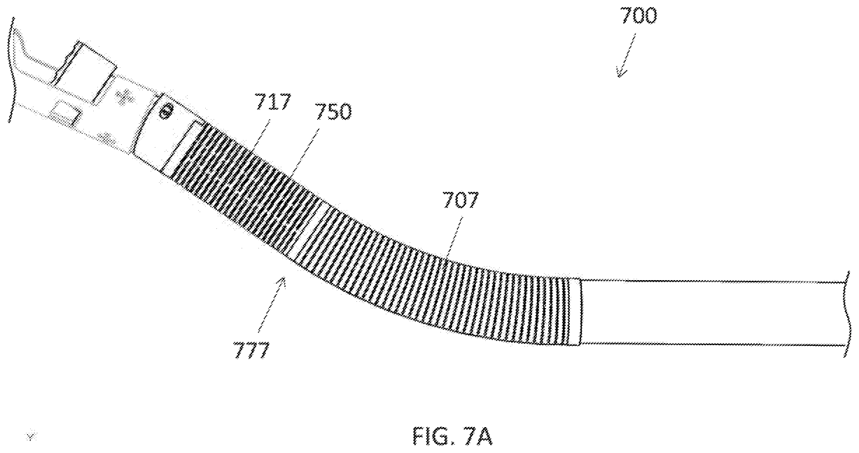

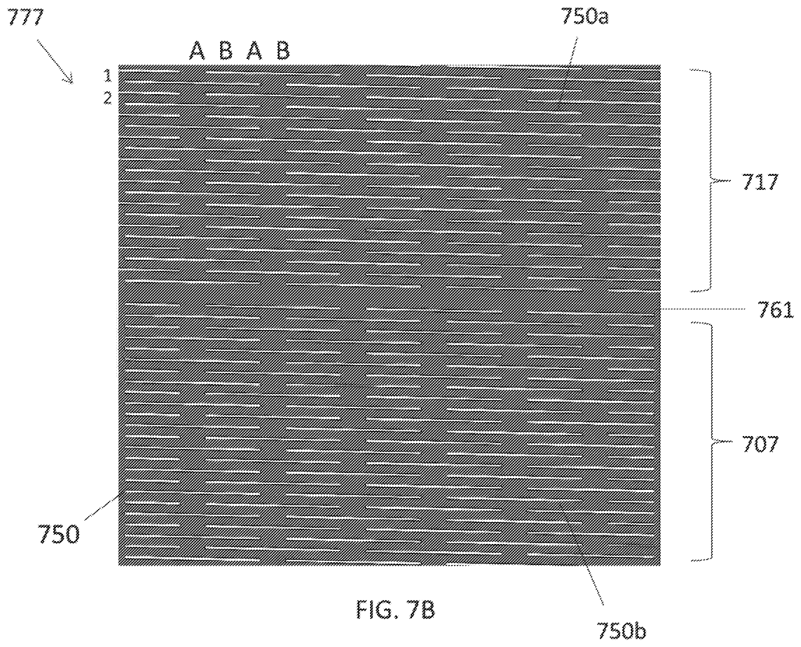

Referring to FIGS. 7A and 7B, in some embodiments, an atherectomy catheter 700 can include a curved portion 777 that includes a fixed jog section 707 and a flexible section 717. The fixed jog section 707 can either be proximal to the flexible section 717 (as shown) or distal to the flexible section 717. In some embodiments, the fixed jog section 707 is longer than the flexible section 717. For example, the fixed jog section 707 can be 5-10 mm, such as 8 mm, and the flexible section 717 can be 2-6 mm, such as 5 mm. Further, in some embodiments (as shown), the fixed jog section 707 can include only a single curve rather than a double curve (e.g., forming a c-shape rather than an s-shape). The angle of the curve can be, for example, 120° to 175° (e.g., 130° to 160°, or approximately) 145°. The flexible section 717 can be configured to bend passively during use (i.e., when acted upon by the vessel wall), for example to form an angle of between 90° and 180°, such as 110-170°, such as 130°-160°.

In some embodiments, the curved portion 777 can be made of a laminated frame. Referring to FIG. 7B, the curved portion 777 can include a frame that includes a plurality of circumferential slits 750a,b extending therethrough. The slits 750a of the flexible section 717 can extend entirely around the circumference (i.e., include no longitudinal spine therein) while the slits 750b of the fixed jog section 707 can end at a longitudinal spine 760 extending through the fixed jog section 707. An annular spine 761 can separate the flexible section 717 and the fixed jog section 707. The frame can be made, for example, of Nitinol or stainless steel. Further, the frame can be laminated with a thin layer of polymer, such as Tecothane, on one or both sides. In some embodiments, only the fixed jog section 707 is laminated while the flexible section 717 remains unlaminated.

Referring to FIG. 7B, the slits 750a,b can be arranged in a pattern that is configured to provide flexibility in the flexible section 717 while maintaining structural integrity of the elongate body in both the flexible section 717 and the fixed jog section 707. Thus, the majority of the slits 750a,b can have the same length, but be offset from one another. For example, the slits 750a in the flexible section 717 can be arranged in rows (1,2) and columns (A, B). Each slit 750a can have a length equivalent to the width of columns A+B+A. Further, the slits can be offset from one another by a distance of A+B. Thus, each column A can include slits from every row 1,2 while column B can include alternating slits (from either row 1 or 2). Column B thus provides structural integrity to the slitted portion of the device. The slits 750a of the flexible section 717 can provide flexibility to allow the catheter 700 to achieve the desired curvature in any direction when inside the body (i.e., the slits can pull apart on the outside of the curve and compress and/or overlap when on the inside of the curve). For example, the flexible section 717 can bend to align the cutter with the edge of the vessel.

Further, the slits 750b in fixed jog section 707 (except the shorter slits bordering the spine 560a) can likewise have a length equivalent to the width of columns A+B+A. Further, the slits can be offset from one another by a distance of A+B. Thus, each column A can include slits from every row 1,2 while column B can include alternating slits (from either row 1 or 2). In fixed jog section 707, however, the spine 760 can be heat-set to set the angle of the jog, fixing the jog.

The curved sections described herein can additionally or alternatively include any of the selective bending support features described in International Application No. PCT/US2019/028415 (the '415 application) (published as WO 2019/204797), which is incorporated by reference herein in its entirety. In some embodiments, the selective bending support features described in the '415 application can be modified to take the s-shape as described herein, such as by including spines on opposite sides of the shaft. Additionally, in some embodiments, the selective bending support features described in the '415 application can be modified so as to be activated by compression (e.g., by pulling on the driveshaft of an atherectomy catheter as described herein) rather than via tension.

In some embodiments, the curved portions of the elongate catheter bodies described herein can form a substantially s-shape with two different inflection points of opposite curvatures. In other embodiments, the curved portion can include a single inflection point that forms substantially a C-shape. Further, in some embodiments, one or more of the curves can be fixed. In other embodiments, one or more of the curves can be user activated (e.g., by pulling on the driveshaft or a separate pullshaft or wire). Further, any of the designs described herein can include a flexible section (e.g., of the elongate body or the nosecone) that allows the catheter to take the desired curvature during use.

In some embodiments, the amount of curvature of the user-adjusted curved portions can be further adjusted either prior to or during an atherectomy procedure based on the curvatures of the artery and the location of the plaque formation. For example, by tensioning a shaft of the catheter, the curved portion can constrict and adopt a sharper angle. Alternatively, when the shaft is relaxed, the curved portion can relax and adopt a wider angle. In such examples, the angles of deflection may be adjusted, for example, by 5 to 20 degrees. Further, the shape and angle can be incrementally and/or continuously adjustable, as described herein.

In some embodiments, the user-adjusted curved portions can have a pre-shaped bend or curvature that can be further adjusted prior to or during an atherectomy procedure. In other embodiments, the curved portions can be straight before the user-activated bend is activated.

In any of the embodiments described herein, the nosecone can be configured to hold tissue that is debulked by the cutter. Further, the driveshaft and cutter can be configured to move distally to pack tissue into the nosecone.

In some embodiments, lamination of a framework can cause the laminating material to heat and shrink, pushing into open slits and fixing the shape of the frame (e.g., in a pre-shaped jog). For example, the curved portions 533 and/or 633 can be laminated so as to create a fixed jog that can either be further adjusted by pulling on the driveshaft or that remains fixed throughout the procedure. In other embodiments, lamination of the framework can keep the slits open and free of material, allowing for greater flexibility.

Although described herein as being activated via compression (e.g., pulling on a driveshaft), the curved portions described herein can alternatively be activated via tension (e.g., pushing on a driveshaft).

The atherectomy catheters having a curved portion described herein advantageously allows easier and closer positioning of the atherectomy cutter to plaque close to the inner artery walls. That is, the curved portions can be configured such that the exposed portion of the cutter (e.g., the area extending through the cutter window) moves closer to the vessel wall than the unexposed side of the cutter. This positioning can make cutting during the atherectomy procedure more efficient.

Any of the curved portions described herein may be used alone or in combination with a mechanism to deflect the nosecone. In some embodiments, the nosecone can be deflected by pulling on a cutter driveshaft. Such deflection mechanisms are described in U.S. patent application Ser. No. 15/072,272, filed Mar. 16, 2016, titled “ATHERECTOMY CATHETERS DEVICES HAVING MULTI-CHANNEL BUSHINGS,” now U.S. Pat. No. 9,592,075; and U.S. patent application Ser. No. 15/076,568, filed Mar. 21, 2016, titled “ATHERECTOMY CATHETERS AND OCCLUSION CROSSING DEVICES,” now U.S. Pat. No. 9/498,247, each of which is incorporated by reference herein in its entirety. In some embodiments, placing further tension on the drive shaft (i.e., after exposing the nosecone) can result in compression being applied to the curved portion, causing the curved portion to assume its final curved configuration. Having both the nosecone deflect and the curved portion can result in better tissue invagination and thus better or more efficient tissue cutting.

In embodiments where the nosecone is not deflected, the respective cutting windows can be optimized so as to allow for automatic invagination of tissue into the cutting window. Further, having the nosecone not deflect and relying entirely on the curved portion for tissue apposition can advantageously prevent the cutter from escaping from the nosecone during packing. Further, having the curved portion alone (i.e., without the nosecone activation) can advantageously eliminate having to use additional mechanisms to force a jog mid-surgery, such as pulling or pushing on a shaft, thereby enhancing both ease of use and enhancing image stability.

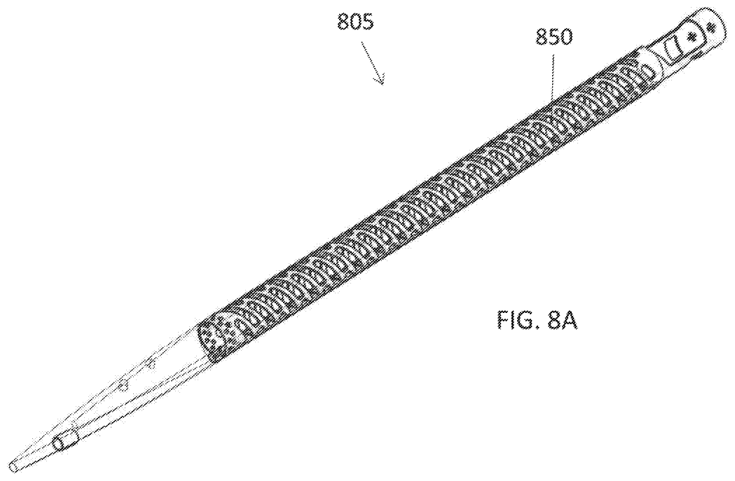

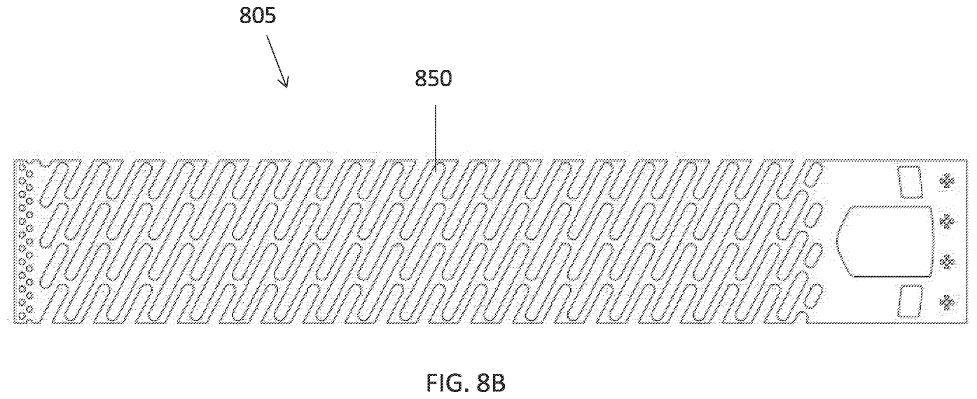

Referring to FIG. 8A, in some embodiments, the nosecone 805 can be flexible. That is, the elongate body can include one or more curves (as described herein), and the nosecone 805 can provide additional flexibility to allow the catheter to take the desired shape. The nosecone 805 can, for example, include a repeating laser cut pattern covered in a laminate layer. As shown in FIG. 8A, the pattern can include a series of spiral slits 850 extending around the circumference of the nosecone. In some embodiments, the laser cut pattern can be cut out of stainless steel, which can be laminated with a polymer, such as Tecothane. Additional flexible nosecone designs are described in U.S. patent application Ser. No. 14/776,749, filed Sep. 15, 2015, titled “TISSUE COLLECTION DEVICE FOR CATHETER,” now U.S. Pat. No. 11,096,717; and International Patent Application No. PCT/US2017/035510, filed Jun. 1, 2017, titled “CATHETER DEVICE WITH DETACHABLE DISTAL END,” published as WO 2017/210466, each of which is incorporated by reference herein in its entirety. The flexible nosecone can be used in addition to, or in place of, any feature of the elongate body curved portions described herein.

Any of the catheter devices described herein can include an imaging system for collecting images outside of the catheter. In some embodiments, the imaging system includes a side-facing optical coherence tomography (OTC) system coupled to a cutter and driveshaft for collecting images outside of catheter while the cutter and driveshaft are rotating. Example suitable imaging systems are described in International Application Nos. PCT/US2017/040431 (published as WO 2018/006041) and PCT/US2019/028415 (published as WO 2019/204797), each of which is incorporated by reference herein in its entirety.

Any of the catheter devices described herein can include a lock assembly for locking an axial position of the driveshaft (inner shaft) relative to the outer shaft (catheter). The lock assembly can be used, for example, to maintain the catheter distal assembly in a curved or straight state or to keep the cutter positioned outside or inside of the cutter window. In some cases, the lock assembly allows for continuous adjustment of the curvature of the catheter as described herein. In some examples, the locking mechanism is in the handle of the catheter device. FIGS. 15A-15C show an example of a slider lock 1501 in a handle 1500 of the catheter device. In FIG. 15A, the slider lock 1501 is in a distal position where a slider button 1503 is most distally positioned. The inner driveshaft includes a hypotube 1513 and a drive key 1515. The drive key 1515 has a square cross-sectional shape that fits within a correspondingly square shaped opening of a distal end of a cradle 1520. This configuration forms a spline joint assembly 1521 that rotationally couples the drive key 1515 to the cradle 1520 but also allows for axial translation of the drive key 1515 relative to the cradle 1520. Thus, when the cradle 1520 is rotated by the drive motor, the inner driveshaft (drive key 1515 and the hypotube 1513) also rotates, while the spline joint assembly 1521 allows for axial translation of the inner driveshaft (drive key 1515 and the hypotube 1513) relative to the cradle 1520 and the drive motor. A connector piece 1510 is positioned around the drive key 1515 and is used to translationally couple the slider button 1503 to the drive key 1515 while allowing the drive key 1515 to rotate independently of the slider button 1503. The connector piece 1510 includes a bearing 1511 (e.g., ball bearing) that allows the drive key 1515 to rotate within the connector piece 1510. The slider button 1503 is rigidly coupled to the connector piece 1510. Thus, distal and proximal movement of the slider button 1503 causes corresponding distal and proximal movement of the inner driveshaft (drive key 1515 and the hypotube 1513). In this way, the spine joint 1521 allows axial translation of the slider button with respect to the driveshaft while allowing the driveshaft to rotate with respect to the other parts of the handle assembly including the slider button.

A curved disc spring 1509 provides resistance against the slider button 1503 to keep teeth 1505 of the slider button 1503 engaged with corresponding teeth 1507 within the housing of the handle 1500, thereby locking an axial position of the driveshaft in place. To move the slider button 1503, a user presses the slider button 1503 radially inward to compress the disc spring 1509 and cause the teeth 1505 of the slider button 1503 to disengage from the teeth 1507 within the housing of the handle 1500, as shown in FIG. 15B. When the slider button 1503 is pressed, the user can slide the slider button 1503 proximally or distally. The slider button 1503 is positioned within an opening of the housing of the handle 1500, where the opening is defined by a distal edge 1517 and a proximal edge 1519 that limit the distal and proximal movement of the slider button 1503. Thus, when the user presses on the slider button 1503, the user can translate the slider button 1503 by any distance between the distal edge 1517 and the proximal edge 1519. When the user releases pressure from the slider button 1503, the disc spring 1509 applies pressure back on the slider button 1503 to reengage the teeth 1505 of the slider button 1503 with the teeth 1507 of the housing of the handle 1500, thereby relocking a position of the driveshaft relative to the non-translating parts of the catheter assembly, including the outer shaft. A user can choose an extent to which the driveshaft is translated within the outer shaft as long as the translation of slider button 1503 is between the distal edge 1517 and the proximal edge 1519. The distance between the distal edge 1517 and the proximal edge 1519 can vary depending on design requirements. In some examples, the distance between distal edge 1517 and the proximal edge 1519 ranges from about 0.5 inches to about 1.5 inches (e.g., 0.5-1.5 inches, 0.5-1.0 inches, 0.5-0.75 inches, or 0.75-1.5 inches, or 0.75-1.0 inches). The pitch of the teeth 1505 and 1507 is associated with a fineness of control that the user has for locking the position of the driveshaft. In some examples, the pitch of the teeth 1505 and 1507 is about 0.030 inches or less (e.g., 0.030 inches, 0.028 inches, 0.026 inches, 0.025 inches, 0.023 inches, 0.020 inches, 0.016 inches, or 0.015 inches or less). In some examples, the pitch of the teeth 1505 and 1507 ranges from about 0.020 inches to about 0.040 inches (e.g., 0.020-0.040 inches, 0.025-0.030 inches, or 0.025-0.040 inches).

FIG. 15C shows the slider lock 1501 locked in a most proximal position where the slider button 1503 is against the proximal edge 1519 of the housing of the handle 1500. As shown, the teeth 1505 of the slider button 1503 are reengaged with the teeth 1507 within the housing of the handle 1500, thereby locking the axial position of the driveshaft (drive key 1515 and the hypotube 1513) relative to other parts of the catheter system, including the outer shaft. Thus, the slider lock 1501 allows the user to move the driveshaft with respect to the outer shaft while the driveshaft is rotating, as well as allowing the user to choose an extent to which the driveshaft is translated within and locked with respect to the outer shaft. In addition, the slider button 1503 allows a selected axial position of the driveshaft to be locked relative to the outer shaft. These features allow the user to select the extent of curvature of the flexible section of the catheter and to lock the flexible section in selected curvature. These features also allow the user to lock the cutter in an active mode (where the cutter extends through the cutter window) or in a passive mode (where the cutter is retracted within the cutter window). For example, having the slider button 1503 in the distal-most position (e.g., FIG. 15A) can equate to the cutter being in a passive position and the flexible section of the elongate body in a straight or unbent position. Moving the slider button 1503 proximally a little from this most-distal position can cause the cutter to pop out of the cutter window. Moving the slider button 1503 still further proximally can cause the flexible section to bend (e.g., in the s-shape curve) with increasing proximal movement of the slider button 1503 causing increasing amounts of curvature. Having the slider button 1503 in the proximal-most position (e.g., FIG. 15C) can equate to the cutter being in an active position and the flexible section of the elongate body being in a maximally curved position. Moving the slider button 1503 distally from this most-proximal position can cause the flexible section to decrease in curvature with increasing distal movement of the slider button 1503 causing decreasing amounts of curvature. Moving the slider button 1503 still further distally can cause the cutter to retract within the cutter window.

FIG. 15C also illustrates portions of a saline flushing system of the catheter. The handle 1500 can include a connector 1535 that is connected to a tube 1503 to provide fluid (e.g., saline) within portions of the catheter. Although not shown, the flexible tubing 1530 can extend (upward in FIG. 15C) to a connector (e.g., luer connector) that allows the user to directly connect a fluid source (e.g., syringe or saline bag). The fluid can flow through the connector 1535 to a fluid housing 1537, which provides fluidic access between the driveshaft and the outer shaft. The fluid can serve several purposes. For example, the fluid can purge air from the catheter, provide lubrication for the rotational movement of the driveshaft, and can displace blood with an optically transparent fluid (e.g., saline) at a distal end of the catheter so that the imaging system can capture images outside of the catheter while in the blood vessel. In this example, the fluid housing 1537 includes a distal end 1532 with a first seal (e.g., O-ring) and a proximal end 1534 with a second seal (e.g., O-ring) that prevent the fluid from entering other regions of the catheter handle assembly, such as the slider lock 1501 region of the handle 1500.

Any of the catheters described herein may include a shapeable section having one or more frames that are configured to provide a predetermined shaped bend. A frame may include an arrangement of articulating features (e.g., slits, spines, backbones and/or rigid members) that allow the frame to take on a predetermined shape. For example, FIGS. 5A-5D shows an exemplary curved portion 533 of a catheter that includes a frame with a series of circumferential slits 550; FIGS. 6A-6E shows another exemplary curved portion 633 having a frame with annular ring spines 661a,b,c connected together by longitudinal spines 660a,b; FIGS. 7A-7B shows another exemplary curved portion 777 having a frame that includes circumferential slits 750a,b; and FIGS. 9A-9F show another exemplary curved portion 933 having slits 947 and opposing longitudinal spines 960a,b configured to form an s-shaped curve. In some cases, the frames are fixed in that they preferentially take on the predetermined shape. For example, the material of the frame may be made of a shape setting material (e.g., nitinol) and set (e.g., heat-set) in the predetermined (e.g., bent) shape. In other cases, the frames preferentially have a substantially straight shape and are configured to take on the predetermined (e.g., bent) shape upon application of a force (e.g., pushing and/or pulling of the inner driveshaft). In some cases, the frames are configured to bend in one lateral direction (i.e., unidirectionally), or to bend in two lateral directions (i.e., bidirectionally).

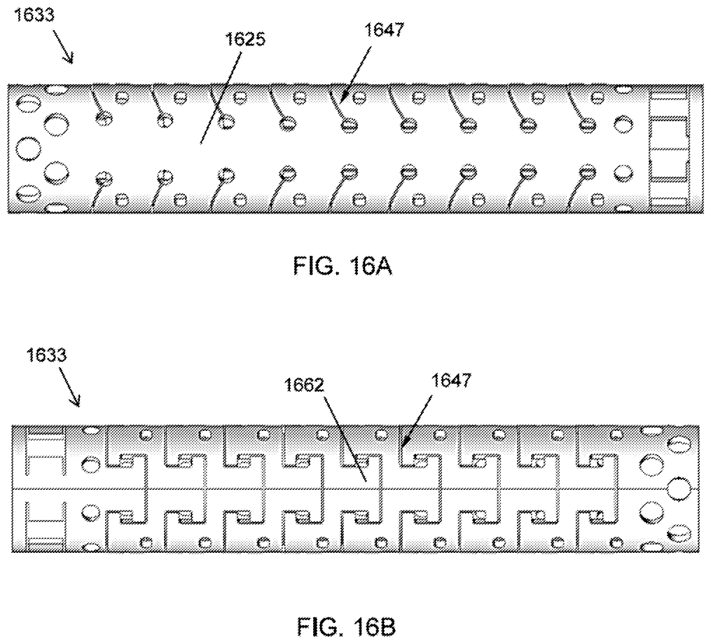

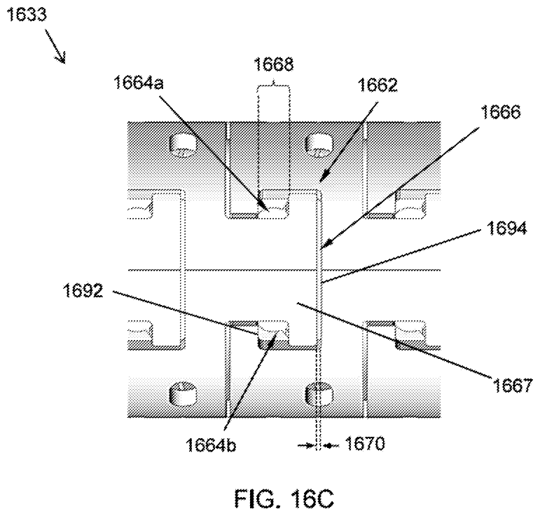

FIGS. 16A-16C show an exemplary tubular frame 1633 that is configured for unidirectional bending, in this case, toward a backbone of the frame. FIG. 16A shows a first side of the tubular frame 1633, showing a pattern of slits 1647 that defines a backbone 1625. FIG. 16B shows a second side (opposite the first side) of the tubular frame 1633a, showing how the pattern of the slits 1647 form bend control features 1662. The bend-control are configured to control the extent to which the frame 1633 bends. As shown in the closeup view of FIG. 16C, each of the bend-control features 1662 includes a T-shaped segment 1667 within a gap of the frame 1633. Each of the bend-control features 1662 includes first spaces 1664a and 1664b on a first side (e.g., distal side) of the T-shaped segment 1667, and a second space 1666 on a second side (e.g., proximal side) of the T-shaped segment 1667. The longitudinal widths 1668 (as measured along the longitude of the frame 1633) of the first spaces 1664a and 1664b dictates how much the frame 1633 can bend in a lateral direction toward the first side of the frame 1633 having the backbone 1625 (i.e., bend toward the backbone 1625). The longitudinal width 1670 (as measured along the longitude of the frame 1633) of the second space 1666 dictates how much the frame 1633 can bend in a lateral direction toward the second side of the frame 1633 opposite the backbone 1625 (i.e., bend away from the backbone 1625).

For example, the first spaces 1664a and 1664b allow room for the second side of the frame 1633 opposite the backbone 1625 to expand the frame 1633 from a straightened (neutral) configuration to a bent configuration toward the backbone 1625 when a distal longitudinal force is applied to frame 1633 (e.g., by pushing the driveshaft). The frame 1633 may be bend toward the backbone 1625 to an extent until the T-shaped segment 1667 contacts a first edge 1692 on the first side (e.g., distal side) of the T-shaped segment 1667, thereby preventing the frame 1633 from further bending toward the backbone 1625. Thus, the first edge 1692 can serve as a stop element to limit lateral bending of the frame 1633 in the direction toward the backbone 1625. The larger the longitudinal widths 1668 of the first spaces 1664a and 1664b, the more the frame 1633 may bend in a direction toward the backbone 1625. A second edge 1694 on the second side (e.g., proximal side) of the T-shaped segment 1667 may prevent the frame 1633 from bending in the direction opposite the backbone 1625. Thus, the second edge 1694 can serve as a stop element to limit lateral bending of the frame 1633 in the direction away from the first side of the frame 1633 having the backbone 1625. Since the longitudinal width 1670 of the second space 1666 is very small, the second side of the frame 1633 opposite the backbone 1625 may contract very little or be prevented from contracting (prevent lateral bending of the frame 1633 away from the backbone 1625). In these ways, the frame 1633 is configured to preferentially bend toward the backbone 1625 with little to no bending away from the backbone 1625.

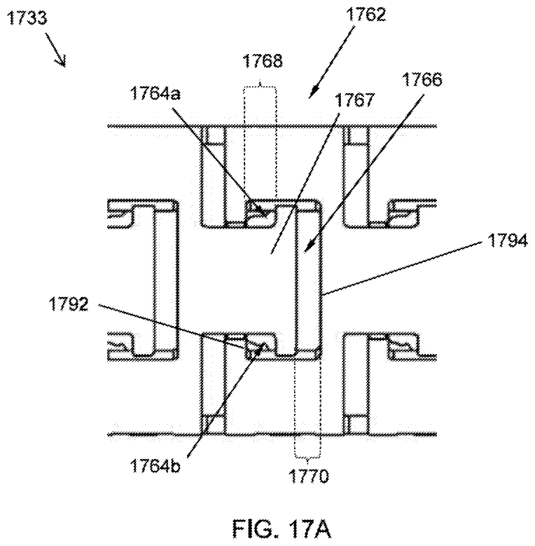

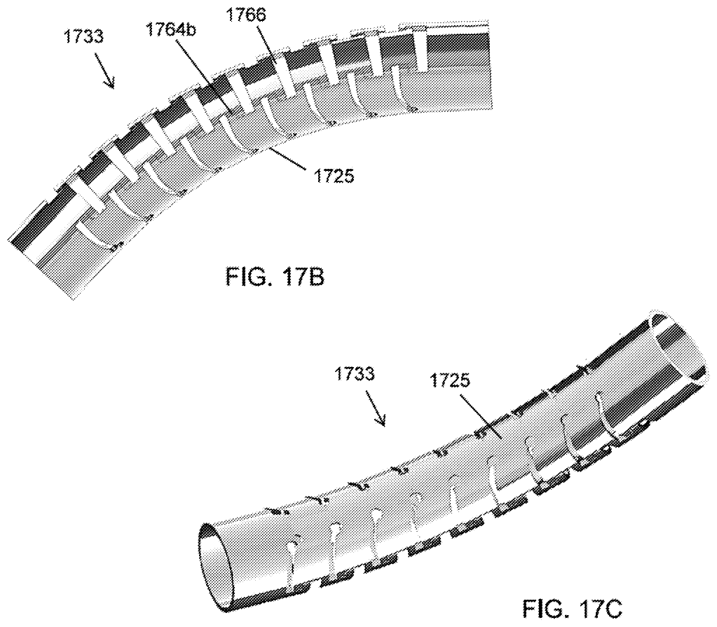

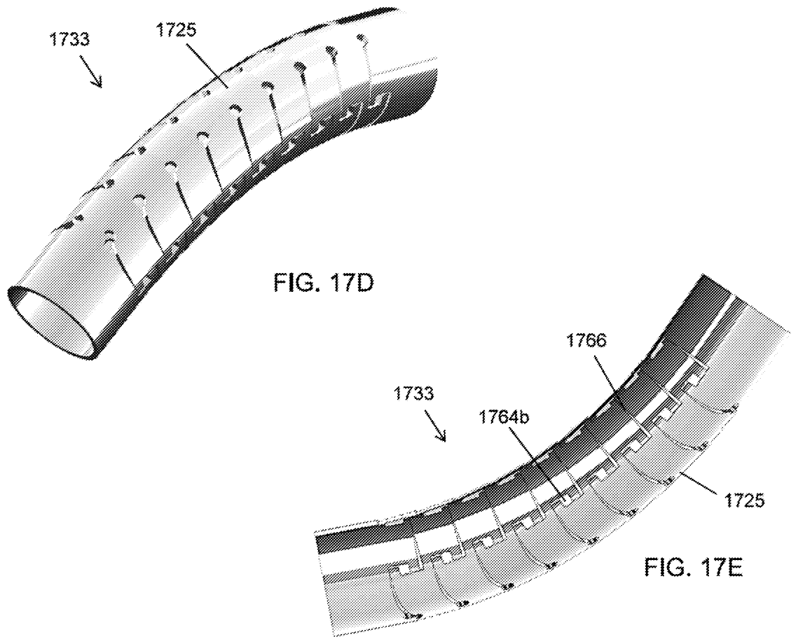

FIGS. 17A-17E illustrate an example frame 1733 having similar bend-control features 1762 as the frame 1633 in FIGS. 16A-16C except that the frame 1733 is configured for bidirectional bending. FIG. 17A shows a closeup view of a second side of the frame 1733 opposite a backbone 1725, illustrating first spaces 1764a and 1764b on a first side (e.g., distal side) and a second space 1766 on the second side (e.g., proximal side) of each T-shaped segment 1767. The longitudinal widths 1768 of the first spaces 1764a and 1764b dictate how much the frame 1733 may bend in a lateral direction toward the first side of the frame 1733 having the backbone 1725 (i.e., bend toward the backbone 1725). The longitudinal width 1770 of the second space 1766 dictates how much the frame 1733 may bend in a lateral direction toward the second side of the frame 1733 opposite the backbone 1725 (i.e., away from the backbone 1725). Compared to the frame 1633, the second space 1766 is large enough to allow the frame 1733 to bend more away from the backbone 1725.

For example, from a straightened (neutral) position of the frame 1733, activation (e.g., by applying a longitudinal force on to frame 1733 (e.g., in the distal direction by pushing the inner driveshaft)), the first spaces 1764a and 1764b allow room for the side of the frame 1733 opposite the backbone 1725 to expand (and the side of the frame 1733 having the backbone 1725 to contract), thereby allowing the frame 1733 to bend toward the backbone 1725 (as shown in FIGS. 17B and 17C). Further pushing of the inner driveshaft can cause the T-shaped segment 1767 to contact a first edge 1792 on a first side (e.g., distal side) of the T-shaped segment 1767, thereby preventing the frame 1733 from further bending. Thus, the first edge 1792 can serve as a stop element to limit lateral bending of the frame 1733 in the direction toward the backbone. A longitudinal force can be applied to the frame 1733 in the proximal direction (e.g., by pulling the driveshaft) to straighten the frame 1733 back to the straightened (neutral) position. When a further longitudinal force is applied to the frame 1733 in the proximal direction (e.g., by pulling the driveshaft), the space 1766 on the second side (e.g., proximal side) of the T-shaped segment 1767 allows the side of the frame 1733 having the backbone 1725 to expand (and the side of the frame 1733 opposite the backbone 1725 to contract), thereby causing the frame 1733 to bend away from the backbone 1725 (as shown in FIGS. 17D and 17E). Further pulling of the driveshaft can cause the T-shaped segment 1767 to contact a second edge 1794 on the second side (e.g., distal side) of the T-shaped segment 1767, thereby preventing the frame 1733 from further bending away from the backbone 1725. Thus, the second edge 1794 can serve as a stop element to limit lateral bending of the frame 1733 in the direction away from the backbone 1725.

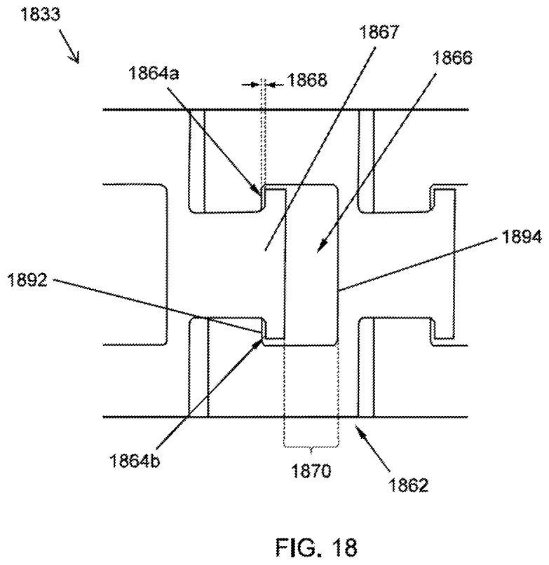

FIG. 18 shows a closeup view of an exemplary tubular frame 1833 that is configured for unidirectional bending, in this case, away from the backbone of the frame. The frame 1833 includes bend-control features (e.g., bending control feature 1862) that includes a T-shaped segment 1867 within a gap of the frame 1833. Each of the bend-control features 1862 includes first spaces 1864a and 1864b on a first side (e.g., distal side) of the T-shaped segment 1867, and a second space 1866 on a second side (e.g., proximal side) of the T-shaped segment 1867. The longitudinal widths 1868 (as measured along the longitude of the frame 1833) of the first spaces 1864a and 1864b dictates how much the frame 1833 can bend in a lateral direction toward the first side of the frame 1833 having the backbone (i.e., bend toward the backbone). The longitudinal width 1870 (as measured along the longitude of the frame 1833) of the second space 1866 dictates how much the frame 1833 can bend in a lateral direction toward the second side of the frame 1833 opposite the backbone (i.e., bend away from the backbone).

For example, the second space 1866 allow room for the second side of the frame 1833 opposite the backbone to compress from a straightened (neutral) configuration to a bent configuration away from the backbone when a proximal longitudinal force is applied to frame 1833 (e.g., by pulling the driveshaft). The frame 1833 may be bend away from the backbone to an extent until the T-shaped segment 1867 contacts a second edge 1894 on the first side (e.g., distal side) of the T-shaped segment 1867, thereby preventing the frame 1833 from further bending away from the backbone. Thus, the second edge 1894 can serve as a stop element to limit lateral bending of the frame 1833 in the direction away from the backbone. The larger the longitudinal width 1870 of the second space 1866, the more the frame 1833 may bend in a direction away from the backbone. A first edge 1892 on the first side (e.g., proximal side) of the T-shaped segment 1867 may prevent the frame 1833 from bending in the direction toward the backbone. Thus, the first edge 1892 can serve as a stop element to limit lateral bending of the frame 1833 in the direction toward the first side of the frame 1833 having the backbone. Since the longitudinal widths 1886 of the first spaces 1864a and 1864b are very small, the frame 1833 may bend very little, or be prevented from bending, in the lateral direction toward the backbone. In these ways, the frame 1833 is configured to preferentially bend away from the backbone with little to no bending toward the backbone.

The shapeable sections of the catheter devices described herein may be configured to take on any of a number of shapes. Advantageously, this may allow a user to position the bent section of the catheter device against inner walls of the blood vessel (i.e., apposition against the vessel walls), thereby providing leverage for the rotating cutter to cut tissue. In some examples, the shapeable section is configured to take on a two-dimensional shape, such as an s-shape or a U-shape. In some examples, the shapeable section is configured to take on a three-dimensional shape (i.e., has as components in three-dimensions (e.g., in x, y, and z directions)). In some examples, three-dimensional shape is a spiral shape.

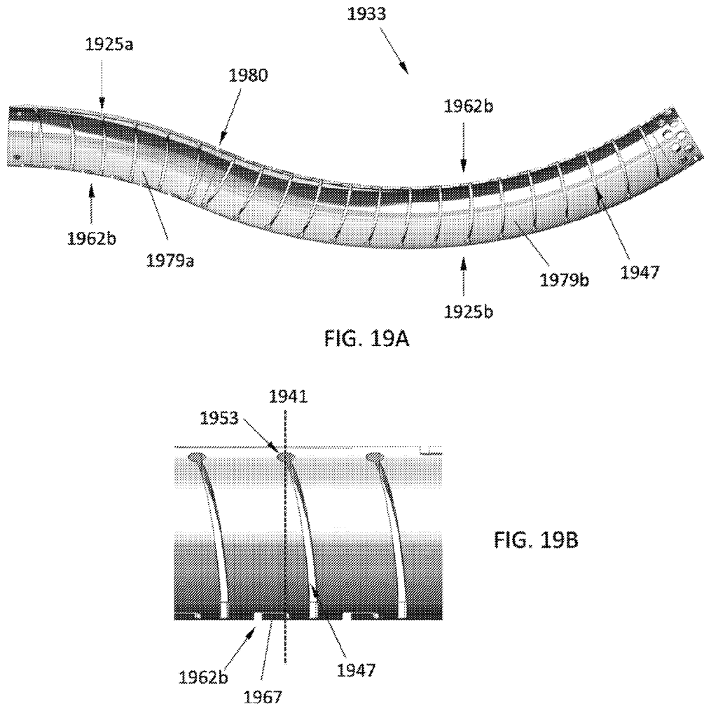

FIGS. 19A and 19B show an exemplary frame 1933 that is configured to bend to an s-shape configuration. The frame 1933 includes a first portion 1979a (e.g., distal portion) and a second portion 1979b (e.g., proximal portion). The first portion 1979a is axially connected to the second portion 1979b at a junction region 1980. In this example, the first portion 1979a includes first bend-control features 1962a configured to bend the first portion 1979a away from a first backbone 1925a, and the second portion 1979b includes second bend-control features 1962b configured to bend the second portion 1979a away from a second backbone 1925b. The first and second bend-control features 1962a, 1962b each have a plurality of T-shaped segments configured to move within corresponding gaps of the frame 1933. In some examples, bending of the first and second portions 1979a, 1979b may be activated by applying a proximal force on the driveshaft therein (e.g., by pulling the driveshaft). As described above, the s-shaped curvature may allow one or both of the first and second portions 1979a, 1979b to butt up against the vessel wall and provide oppositional force(s) for the cutter to contact and cut tissue. Thus, the first and second portions 1979a, 1979b, when bent, may provide two potential areas of contact for the frame 1933 with the vessel.

In some examples, each of the first and second bend-control features 1962a, 1962b are configured for unidirectional bending away from respective backbones 1925a, 1925b, such as described above with respect to FIG. 18. In some examples, one or more of the first and second bend-control features 1962a, 1962b is configured for bidirectional bending toward and away from respective backbones 1925a, 1925b, such as described above with respect to FIG. 17A. In cases where the frame 1933 is configured for bidirectional bending, the first and second bend-control features 1962a, 1962b may be configured to bend more away from the backbones 1925a, 1925b compared to toward the backbones 1925a, 1925b.

As shown in the closeup side view of FIG. 19B, in this example, the frame 1933 includes slits 1947 that are in a slanted arrangement with respect to a transverse axis 1941 when the frame 1933 is in a neutral (e.g., straight) configuration. This slanted slit arrangement allows for a pivot point 1953 associated the bend-control feature 1962b to be radially aligned (or close to radially aligned) with a least a portion of the corresponding bend-control feature 1962b, thereby minimizing an extent to which the features of the bend-control feature 1962b (T-shaped segment 1967) protrude with respect to a curvature of the frame 1933 when bent.

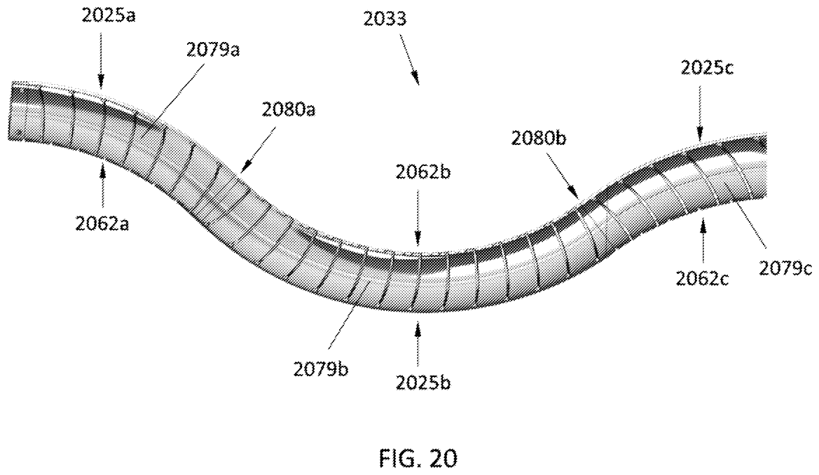

FIG. 20 shows an exemplary frame 2033 that is similar to the frame 1933 of FIGS. 19A and 19B, except that the frame 2033 includes three axial portions 2079a, 2079b, and 2079c that are arranged to bend the frame 2033 in U-shape. The frame 2033 includes a first portion 2079a (e.g., distal portion) having a first a backbone 2025a and plurality of bend-control features 2062a, a second portion 2079b (e.g., middle portion) having a second a backbone 2025b and plurality of bend-control features 2062b, and a third portion 2079c (e.g., proximal portion) having a third a backbone 2025c and plurality of bend-control features 2062c. The first portion 2079a is axially connected to the second portion 2079b at a first junction region 2080a, and second portion 2079b is axially connected to the third portion 2079c at a second junction region 2080b.

When the frame 2033 is activated (e.g., by applying a proximal force on the driveshaft therein (e.g., by pulling the driveshaft)), each of the first, second, and third portions 2079a, 2079b, and 2079c bend, thereby forming a first curve (of the first portion 2079a), a second curve (of the second portion 2079b) and third curve (of the third portion 2079c). The U-shaped curvature may allow one or more of the first, second, and third portions 2079a, 2079b, and 2079c to butt up against the vessel wall and provide oppositional force(s) for the cutter to contact and cut tissue. Thus, the first, second, and third portions 2079a, 2079b, and 2079c, when bent, may provide three potential areas of support for the frame 1933 within the vessel.

In some examples, the first, second, and third portions 2079a, 2079b, and 2079c are configured for unidirectional bending away from respective backbones 2025a, 2025b, and 2025c such as described above with respect to FIG. 18. In some examples, the first, second, and third bend-control features 2062a, 2062b, and 2062c are configured for bidirectional bending toward and away from respective backbones 2025a, 2025b, and 2025c, such as described above with respect to FIG. 17A. In cases where the frame 2033 is configured for bidirectional bending, the first, second, and third portions 2079a, 2079b, and 2079c may be configured to bend more away from the backbones 2025a, 2025b, and 2025c compared to toward the backbones 2025a, 2025b, and 2025c.

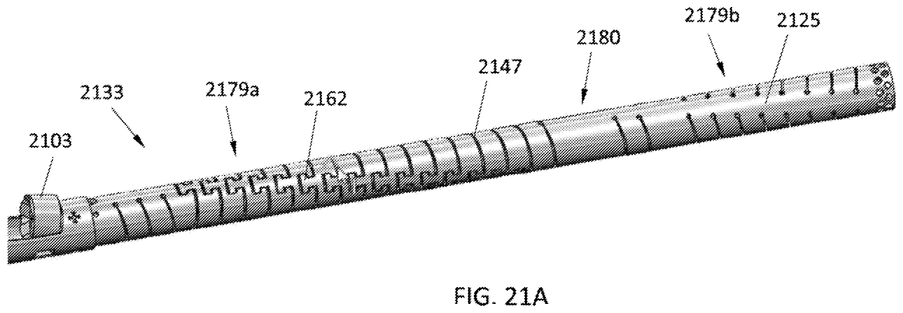

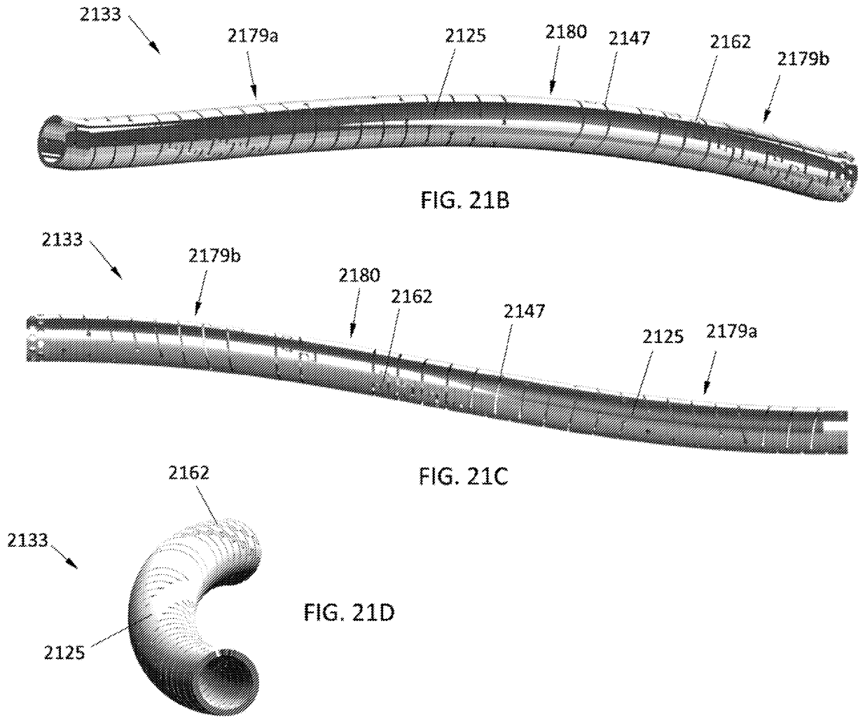

FIGS. 21A-21D show an exemplary frame 2133 that is configured to twist into a spiral shape up to 180 degrees. FIG. 21A shows the frame 2133 in an uncurved (e.g., straight) configuration, and FIGS. 21B-21D shows the frame 2133 in a curved configuration, in this case, twisted by 180 degrees. The frame 2133 (and the shapeable section of the catheter) is axially aligned along a long axis when in the uncurved configuration, curved in multiple planes relative to the long axis when in the curved configuration. For example, the frame 2133 may be configured to curve in three planes (x, y, z) relative to the long axis of the frame. The frame 2133 includes a backbone 2125 and plurality of bend control features 2162 that wind longitudinally 180 degrees around the frame 2133. As shown, the backbone 2125 remains on an opposing side of the frame as the bend control features 2162 as they wind around the frame 2133. The bend control features 2162 and/or the backbone 2162 may spiral around the frame 2133. In this example, the frame 2133 includes a first axial portion 2179a (e.g., distal portion) that is coupled to a second axial portion 2179b (e.g., proximal portion) at a junction region 2180. The junction region 2180 may not include slits 2147 or bend control features 2162, and therefore may not be configured to bend/twist.

Bending/twisting of the first and second portions 2179a and 2179b may be activated by applying a proximal force on the driveshaft therein (e.g., by pulling the driveshaft). In this example, the frame 2133 is configured to twist by up to 180 degrees. The spiral-shaped curvature provides a three-dimensional shape, which may provide more potential areas of contact of the catheter devices against the inner walls of the blood vessel for providing oppositional force(s) for the cutter 2103. That is, one or more portions of the shapeable portion (e.g., including the frame 2133) of the catheter when in the curved configuration may be pressed against the inner walls of a blood vessel to provide an opposing force for efficient cutting of the tissue.

In some examples, the first and second portions 2179a and 2179b are configured for unidirectional bending away from the backbone 2125 such as described above with respect to FIG. 18. In some examples, the first and second portions 2179a and 2179b are configured for bidirectional bending toward and away from the backbone 2125, such as described above with respect to FIG. 17A. In cases where the frame 2133 is configured for bidirectional bending, the first and second portions 2179a and 2179b may be configured to bend more away from the backbone 2125 compared to toward the backbone 2125.

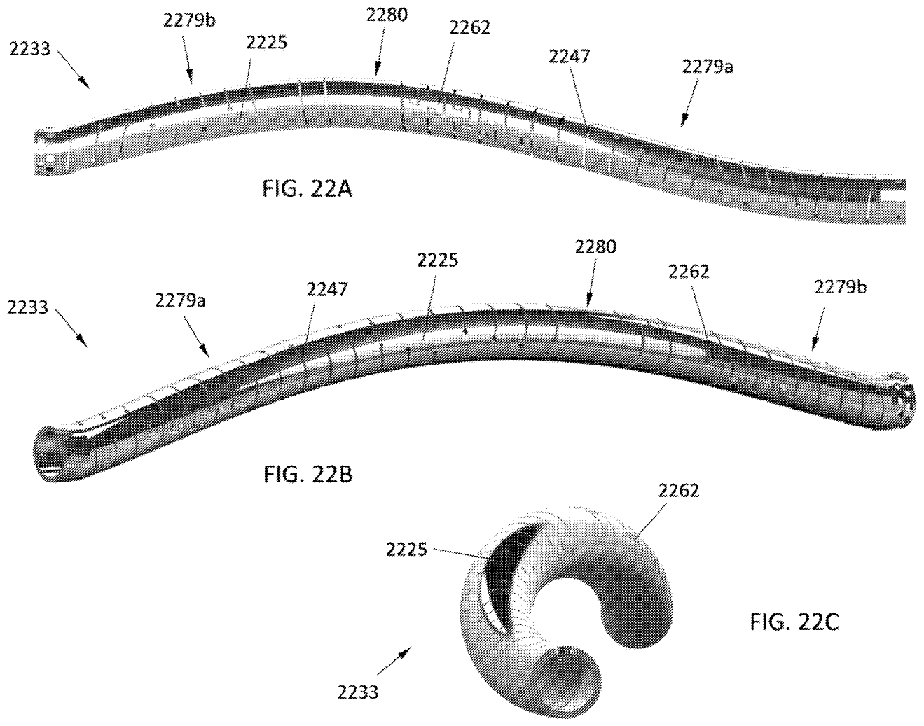

FIGS. 22A-22C show an exemplary frame 2233 that is similar to the frame 2133 of FIGS. 21A-22C, except that the frame 2233 is configured to twist into a spiral shape up to 270 degrees. The frame 2233 includes a backbone 2225 and plurality of bend control features 2262 that wind longitudinally 270 degrees around the frame 2233. As shown, the backbone 2225 remains on an opposing side of the frame as the bend control features 2262 as they wind around the frame 2233. In this example, the frame 2233 includes a first axial portion 2279a (e.g., distal portion) that is coupled to a second axial portion 2279b (e.g., proximal portion) at a junction region 2280. Bending/twisting of the first and second portions 2279a and 2279b may be activated by applying a proximal force on the driveshaft therein (e.g., by pulling the driveshaft). Such 270-degree twisting may allow for a different type of support for the catheter device against the blood vessels compared to the 180-degree twisting configuration of the frame 2133 of FIGS. 21A-21C.

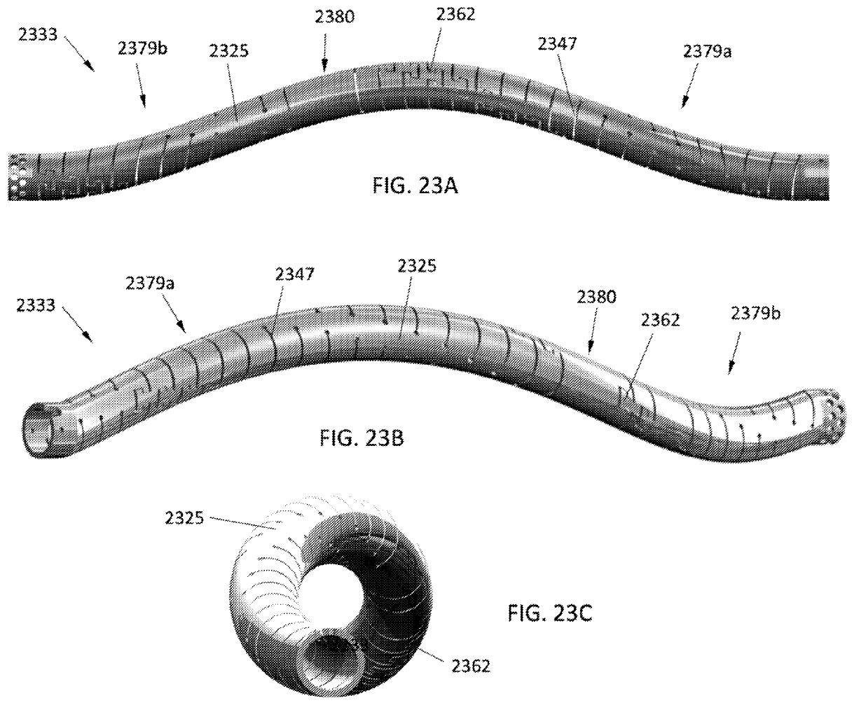

FIGS. 23A-23C show an exemplary frame 2333 that is similar to the frames 2133 and 2233 (FIGS. 21A-22C and 22A-22C), except that the frame 2333 is configured to twist into a spiral shape up to 360 degrees. The frame 2333 includes a backbone 2325 and plurality of bend control features 2362 that wind longitudinally 360 degrees around the frame 2333. As shown, the backbone 2325 remains on an opposing side of the frame as the bend control features 2362 as they wind around the frame 2333. In this example, the frame 2333 includes a first axial portion 2379a (e.g., distal portion) that is coupled to a second axial portion 2379b (e.g., proximal portion) at a junction region 2380. Bending/twisting of the first and second portions 2379a and 2379b may be activated by applying a proximal force on the driveshaft therein (e.g., by pulling the driveshaft). Such 360-degree twisting may allow for a different type of support for the catheter device against the blood vessels compared to the 180-degree twisting configuration of the frame 2133 of (FIGS. 21A-21C) and the 270-degree twisting configuration of the frame 2233 of (FIGS. 22A-22C).

The shapable sections of any of the catheter devices described herein may be combined with any other aspects related to operation of the catheter devices described herein. For example, forces applied to the driveshaft may be used to activate the shapable section and to separately move the cutter, as described herein. For instance, applying a force on the driveshaft (e.g., in the proximal direction (e.g., by pulling the driveshaft)) may cause activation (e.g., bending) of the shapeable section, and applying an opposite force on the driveshaft (e.g., in the distal direction (e.g., by pushing the driveshaft)) may cause the shapeable section to straighten (and/or bend in an opposing direction). In some examples, once the shapeable section is in a bent configuration, movement of the driveshaft in the proximal direction (e.g., by pulling the driveshaft) may cause the cutter to tilt away from the catheter and extend through the cutter window. In some examples, once the shapeable section is in a straight configuration (or bend in an opposing direction), movement of the driveshaft in the distal direction (e.g., by pushing the driveshaft) may cause the cutter to tilt back toward the catheter and retract within the cutter window. Further distal movement of the driveshaft (e.g., by pushing the driveshaft) may cause the cutter to extend distally into the nosecone (e.g., for packing tissue).

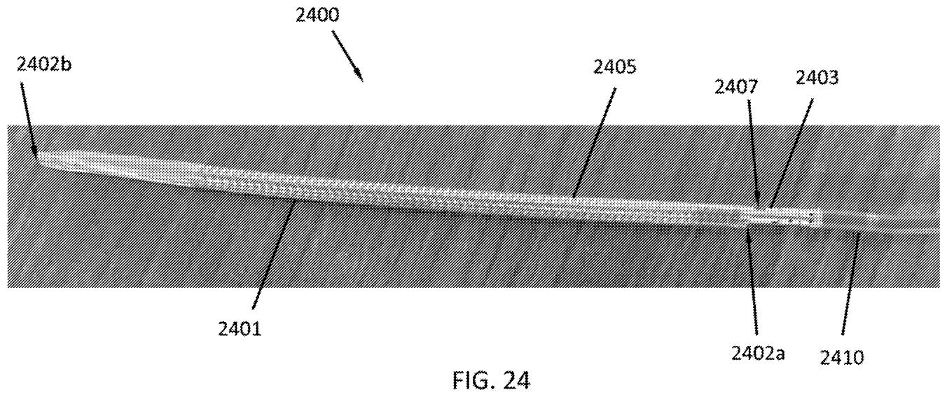

Any of the catheter devices described herein may include a guidewire lumen for accepting a guidewire. FIG. 24 shows distal portion of an exemplary atherectomy catheter 2400 having a guidewire lumen 2401 that is sized and shaped to accept a guidewire therein. The guidewire may be used to transport the catheter 2400 to a target location within the patient's blood vessel. In this example, the guidewire lumen 2401 extends from a first opening 2402a to a second opening 2402b longitudinally along a nosecone 2405 of the device 2400. As described above, the nosecone 2045 may include a reservoir for storing cut tissue. In some examples, the cutter is configured to move distally at least partially into the nosecone 2045 for packing the tissue therein. The guidewire lumen 2401 may be offset with respect to central axis of the nosecone 2405. The guidewire lumen 2401 may run along a side of the catheter 2400 that is opposite the window 2407 for the rotatable cutter 2403. As shown, the nosecone 2405 (with the guidewire lumen 2401) and the window 2407 are distally located with respect to an elongate body 2410 of the catheter 2400. As described herein, the elongate body 2410 may include a shapeable portion (e.g., including any one of the frames and/or flexible portions described herein). The guidewire may be configured to run adjacent to the elongate body 2410. For example, the elongate body 2410 may not include a guidewire lumen.

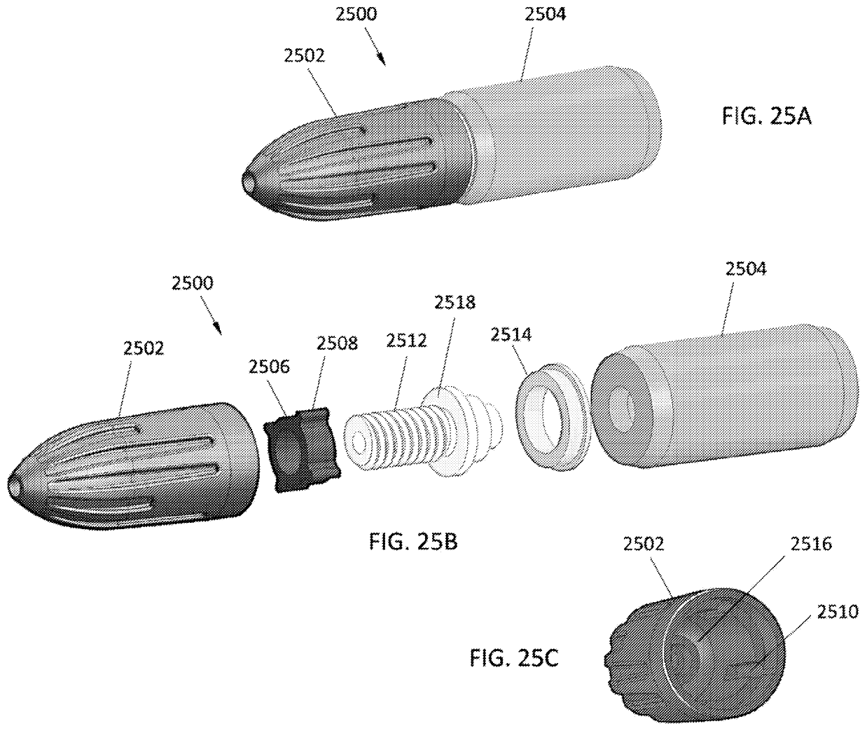

The handle at the proximal portion of the catheter 2400 may be used to rotate the catheter 2400 relative to the handle. This will help a user to maneuver the catheter 2400 within the patient's blood vessel without having to rotate the entire handle. However, rotating the catheter 2400 too many times in one direction may cause the guidewire (within the guidewire lumen 2401) to become twisted or looped. FIGS. 25A-25C shows an exemplary rotation limiter 2500 configured to limit the number of rotations that a catheter (e.g., catheter 2400) can be rotated with respect to the guidewire, thereby preventing such twisting of the guidewire. The rotation limiter 2500 may be located at a distal portion of the handle (See, e.g., rotation limiter 2500 in FIGS. 15A-15C). In this case, the rotation limiter 2500 includes a knob 2502, a proximal body 2504, a nut 2506, a threaded member 2512, and a washer 2514. The knob 2502 and the proximal body 2504 cooperate to form a housing that houses the nut 2506, the threaded member 2512, and the washer 2514. The knob 2502 is configured to rotate (e.g., by a user's hand) the catheter 2400 relative to the proximal body 2504 and the remainder of the handle. This way, the user may rotate the catheter 2400 while maneuvering the catheter 2400 within the patient's blood vessel without rotating the entire handle. The nut 2506 includes wings 2508 that correspond to radially outward protrusions, which are shaped and sized to be captured within recessed track 2510 within the inner surface of the knob 2502. The nut 2506 also includes a central opening having threads that correspond to the outer threads of the threaded member 2512. When the knob 2502 is rotated, the nut 2506 is configured to translate axially along the threaded member 2512 such that the wings 2508 of the nut 2506 translate axially along the recessed track 2510 of the knob 2502. A first stop surface 2516 within the knob 2502 limits the amount of distal movement of the nut 2506 within the recessed track 2510. A second surface 2518 of the threaded member 2512 limits the amount of proximal movement of the nut 2506 within the recessed track 2510. Thus, the wings 2508 remain trapped within the recessed track 2510 and the nut 2506 remains coupled with the knob 2502. In this way, the number of rotations of the knob 2502 is limited by the translational constraints of the nut 2506. In some examples, the knob 2502 is configured to rotate a total of 4 rotations (e.g., two in a first direction (e.g., clockwise) and two in a second direction (e.g., counterclockwise)). However, the rotation limiter 2500 may be configured to rotate any number of total rotations (e.g., 6, 5.5, 5, 4.5, 4, 3.5, 3, 2.5, 2, 1.5, 1, or 0.5 rotations).

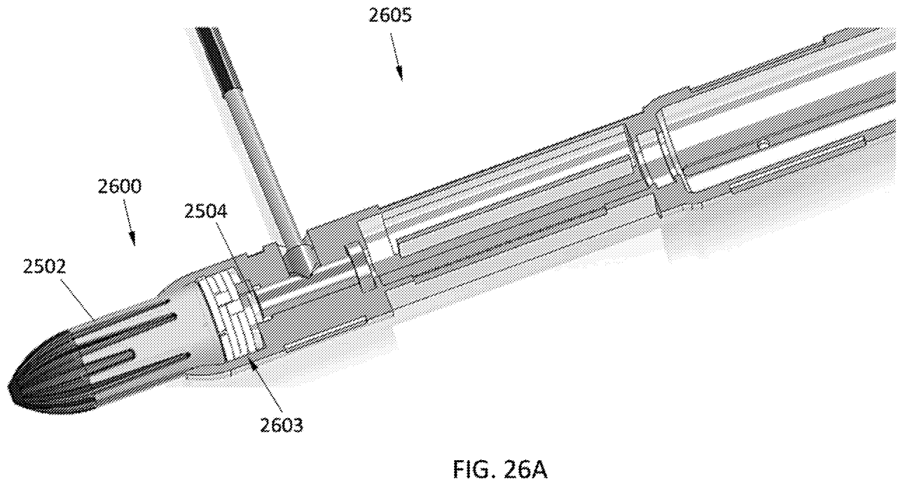

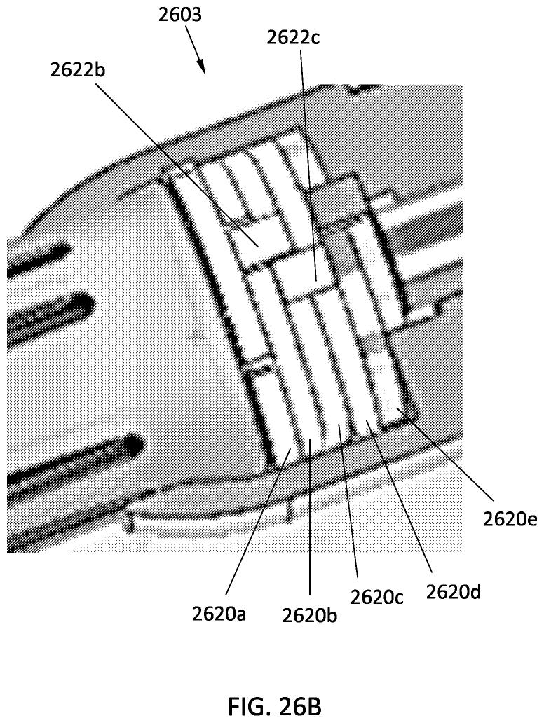

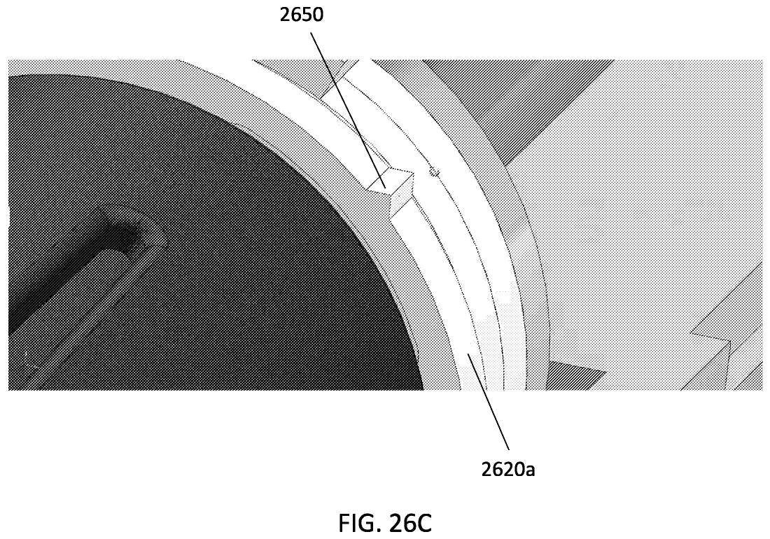

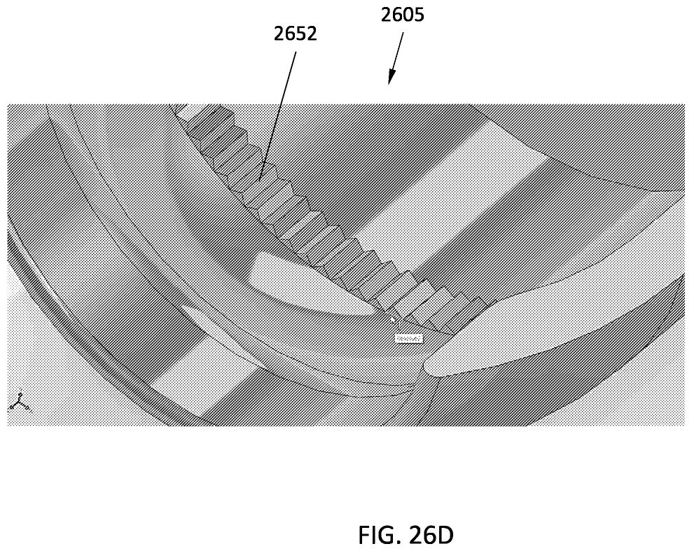

FIGS. 26A-26D show a handle 2605 having another exemplary rotation limiter 2600 with similar features as the rotation limiter 2500 of FIGS. 25A-25C, except that the rotation limiter 2600 includes a disk assembly 2603 that is configured to limit the number of rotations of a knob 2602 relative to a proximal body 2604. As shown in the closeup of FIG. 26B, the disk assembly 2603 includes a series of axially arranged disks 2620a, 2620b, 2620c, 2620e, and 2620e. A first disk 2620a is fixedly coupled to the rotatable knob 2502, a last disk 2620e is fixedly coupled to a proximal body 2604, and intervening disks 2620b, 2620c and 2620d are floating. The disks are 2620a, 2620b, 2620c, 2620e, and 2620e are rotationally linked in that they each are configured to limit rotation with respect to an adjacent disk. For example, a second disk 2620b includes a second radial protrusion 2622b that, when rotated, circumferentially abuts a third radial protrusion 2622c of a third disk 2602c. Thus, each pair of disks provides about one turn of rotation (about 360-degree rotation). In this case, the stack includes five disks 2620a, 2620b, 2620c, 2620e, and 2620e, thereby allowing for about four rotations (e.g., about two in one direction and about two in a second direction). Although in this example the rotation limiter 2600 is configured to allow about four rotations, the rotation limiters described herein may be configured to allow rotation any number (e.g., 6, 5.5, 5, 4.5, 4, 3.5, 3, 2.5, 2, 1.5, 1, or 0.5 rotations). In some cases, the disk assembly rotation limiter 2600 may result in less internal stress compared to the nut assembly rotation limiter 2500 of FIGS. 25A-25C.

FIGS. 26C and 26D show closeup views of a feedback feature of the rotation limiter 2600. As shown in FIG. 26C, the first disk 2620a includes one or more teeth 2650 that radially protrude from an outer circumferential surface of the disk 2620a. In this case, the tooth 2650 has a V-shape. FIG. 16D shows an internal surface of the handle 2605 (e.g., proximal body 2604), showing a gear track 2652 for the tooth 2650 to engage with (e.g., click). Engagement of the one or more teeth 2650 with the corresponding shaped elements of gear track 2652 may provide tactile and/or audible feedback as the disk assembly 2603 is rotated by a user. This feedback feature may provide a rachet-like “feeling” and/or sound for the user.

An extent to which any of the shapeable sections of the catheter devices described herein may be controlled by the handle (See e.g., FIGS. 15A-15C, 25A-25C and/or 26A-26D). For example, an amount of curvature of the shapeable section may be selected by an amount of axial force applied to the driveshaft at the handle. For instance, a greater force applied to the driveshaft (e.g., in the proximal direction) may cause the shapeable section to bend to a further extent. A maximum curvature of the shapeable section may be limited, for example, by the bend-control features described herein.

It should be understood that any features described herein with respect to one embodiment can be combined with or substituted for any feature described herein with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

The terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

A complete listing of the claims follows:

1. An atherectomy device comprising:
a catheter including a distal nosecone coupled to an elongate body, the catheter including a cutter window between the nosecone and the elongate body, wherein the elongate body includes a shapeable section including a first portion, a second portion, and a third portion each configured to bend upon activation; and
a driveshaft configured to rotate and translate within the catheter, the driveshaft including a distal cutter configured to extend through the cutting window, wherein a force applied to the driveshaft in a proximal direction causes the shapeable section to take on a U-shape defined by a first curve of the first portion, a second curve of the second portion, and a third curve of the third portion.

2. The atherectomy device of claim 1, wherein the nosecone is fixedly coupled to the elongate body at a fixed bend of the catheter.

3. The atherectomy device of claim 2, wherein the cutter window is on a convex side of the fixed bend.

4. The atherectomy device of claim 2, wherein the cutter window is distally located along the catheter with respect to the fixed bend.

5. The atherectomy device of claim 1, wherein an extent of curvature of the shapeable section is adjustable based on an amount of force applied to the rotatable driveshaft in the proximal direction.

6. The atherectomy device of claim 1, wherein the cutter is configured to tilt in a first direction and extend though the cutter window upon proximal movement of the driveshaft with respect to the catheter.

7. The atherectomy device of claim 6, wherein the cutter is configured to tilt in a second direction opposite the first direction and retract within the cutter window upon distal movement of the driveshaft with respect to the catheter.

8. The atherectomy device of claim 7, wherein the cutter is configured to extend distally within the nosecone upon further distal movement of the driveshaft with respect to the catheter.

9. The atherectomy device of claim 1, wherein the shapeable section is configured to revert back to a straight configuration upon distal movement of the driveshaft.

10. The atherectomy device of claim 9, wherein the shapeable section includes a frame configured to limit an extent to which the shapeable section bends.

11. The atherectomy device of claim 1, wherein the shapeable section includes a first axial portion, a second axial portion, and a third axial portion, wherein the first and third axial portions are configured to bend in a first direction, wherein the second axial portion is configured to bend in a second direction opposite the first direction.

12. The atherectomy device of claim 11, wherein each of the first, second and third axial portions includes a longitudinal backbone, wherein activation of the shapeable section causes each of the first, second and third axial portions to bend away from its corresponding backbone.

13. The atherectomy device of claim 1, wherein the shapeable section includes a tubular frame having a plurality of slits, wherein activation of the shapeable section causes compression of the tubular frame such that spaces between the plurality of slits become closer together.

14. The atherectomy device of claim 13, wherein the slits are slanted with respect to a transverse axis of the tubular frame.

15. The atherectomy device of claim 1, wherein the shapeable section includes a tubular frame having bend control features configured to limit an extent to which the shapeable section bends.

16. The atherectomy device of claim 1, wherein the first, second and third curves are arranged along a plane.

17. The atherectomy device of claim 1, wherein the shapeable section includes portions that are configured to bend in at least two different lateral directions.

18. The atherectomy device of claim 1, wherein the cutter includes an imaging sensor configured to collect images outside of the catheter while the cutter rotates.

19. A method of using an atherectomy device, the atherectomy device including a cutter coupled to a rotatable driveshaft within a catheter, the catheter having a distal nosecone coupled to an elongate body and a cutter window between the distal nosecone and the elongate body, the method comprising:
applying an axial force in a proximal direction on the driveshaft within the catheter to cause a shapeable section of the elongate body to form a U-shape defined by a first curve, a second curve and a third curve.

20. The method of claim 19, further comprising moving the driveshaft in a proximal direction to cause the cutter to tilt in a first direction and extend through the cutter window.

21-105. (canceled)
